(19) United States
(12) Patent Application Publication
(10) Pub. No.: US 2024/0271226 A1
(43) Pub. Date: Aug. 15, 2024

(54) METHODS OF PATHOGEN IDENTIFICATION AND ANTIMICROBIAL SUSCEPTIBILITY TESTING

(71) Applicant: THE JOHNS HOPKINS UNIVERSITY, Baltimore, MD (US)

(72) Inventors: Tza-Huei Jeff WANG, Germantown, MD (US); Aniruddha Mrithinjay KAUSHIK, Cincinnati, OH (US); Kuangwen HSIEH, Baltimore, MD (US)

(73) Assignee: THE JOHNS HOPKINS UNIVERSITY, Baltimore, MD (US)

(21) Appl. No.: 18/245,048

(22) PCT Filed: Sep. 29, 2021
(86) PCT No.: PCT/US2021/052581

§ 371 Date: Mar. 13, 2023

Related U.S. Application Data
(60) Provisional application No. 63/086,311, filed on Oct. 1, 2020.

(51) Int. Cl. C12Q 1/689; B01L 3/00; C12Q 1/6818; C12Q 1/6897

(52) U.S. Cl. CPC C12Q 1/689; B01L 3/502784; C12Q 1/6818; C12Q 1/6897; B01L 2200/0673; B01L 2200/16; B01L 2300/0663; B01L 2300/0829; B01L 2300/0883

(57) ABSTRACT

Provided herein are methods of identifying and determining the antimicrobial susceptibility of bacteria in samples. Related devices, systems, reaction mixtures, kits, and other methods are also provided.

### CROSS-REFERENCE TO RELATED APPLICATIONS

This application is the national stage entry of International Patent Application No. PCT/US2021/052581, filed on Sep. 29, 2021, and published as WO 2022/072448 A1 on Apr. 7, 2022, which claims the benefit of U.S. Provisional Patent Application Ser. No. 63/086,311, filed on Oct. 1, 2020, both of which are hereby incorporated by reference herein in their entireties.

### STATEMENT REGARDING FEDERALLY SPONSORED RESEARCH OR DEVELOPMENT

This invention was made with government support under grants AI117032 and AI137272 awarded by the National Institutes of Health. The government has certain rights in the invention.

### BACKGROUND

Urinary tract infection (UTI) is one of the most prevalent infectious diseases in the world, affecting 50-60% of women at least once in their lifetime (1-3). In the United States, UTIs account for over 2 million emergency department visits and approximately $3.5 billion in healthcare costs annually (inflation-adjusted to 2020) (4-6). Definitive and clinically actionable diagnoses for UTIs, including both pathogen identification (ID) and antimicrobial susceptibility testing (AST), are currently based on traditional culture-based methods and thus require several days to complete. Consequently, UTIs are often empirically treated at outpatient clinics with first-line, broad-spectrum oral antibiotics. Overuse and misuse of antibiotics through such empiric antimicrobial treatment have contributed to the recent rise in antimicrobial resistant pathogens (7-9). For example, in Baltimore, USA, the resistance rate of uropathogenic E. coli to the first-line antibiotic ciprofloxacin is as high as 31%, and in communities across the world, this rate is steadily increasing (10-12). The burden of increasing antimicrobial resistance is ultimately borne by the patient, as reflected by increasing UTI-related hospitalizations, patient morbidity, and associated costs (6, 13).

Unlike empiric treatment, evidence-based antimicrobial treatment can significantly improve patient outcomes and curtail antimicrobial resistance (14, 15). However, adoption of evidence-based treatment for UTIs in clinical settings remains contingent upon the development of a new diagnostic platform that can provide both pathogen ID and AST results well within 60 minutes following specimen collection (16, 17). Importantly, because ˜90% of UTIs are caused by the Gram-negative species Escherichia coli (˜75% of UTIs), Proteus mirabilis, Klebsiella pneumoniae, and other members of the taxonomic order Enterobacterales (formerly members of the Enterobacteriaceae family (18)), the new platform should appropriately classify these species to establish clinical prognosis and facilitate pathogen-specific antimicrobial therapy. Similarly, the new platform should perform AST on at least first-line antibiotics in order to ensure appropriate therapy, safeguard these first-line antibiotics, and support antimicrobial stewardship (19, 20). Finally, to operate within a clinically relevant time frame and to improve the prospect for clinical adoption, the new platform should allow direct urine sample-to-answer analysis without time-consuming and labor-intensive sample preparation steps such as clinical isolation or nucleic acid extraction. It is therefore desirable that new UTI diagnostic platforms can simultaneously meet these important yet challenging conditions.

Recently-developed UTI diagnostic assays and technologies have generally relied on either nucleic acid amplification tests (NAATs) for pathogen ID or phenotypic measurements for AST. Indeed, diagnostic assays based on PCR (the most mature of the NAATs) have become turnkey for pathogen ID (21-24) but provide little AST information. Similarly, phenotypic measurements of bacterial cells—exemplified by microfluidic- and single-cell-based strategies such as microscopy (25-27), Raman spectroscopy (28, 29), or microcantilevers (30, 31)—have accelerated AST to as short as <1 hour but still lack accurate pathogen ID. These shortcomings have prompted the development of “pheno-molecular AST”—an approach that combines quantitative, nucleic acid-based molecular detection of bacteria and growth-based (i.e., phenotypic) AST (32-38). In pheno-molecular AST, bacteria are briefly grown in the presence and absence of antibiotics; the amounts of bacterial nucleic acids—serving as surrogates of phenotypic responses to antibiotics—are then quantitatively detected in each case and compared to reveal antibiotic susceptibilities. Pathogen ID is readily achieved via species-specific detection probes or post-amplification analysis (e.g., high-resolution DNA melt curve analysis (33, 34)). To date, the most advanced pheno-molecular AST platform can specifically detect a single pathogen (i.e., E. coli) and complete AST against a single antibiotic (e.g., ciprofloxacin) within 30 minutes (38). Though a significant advance, this platform still lacks the ability to identify or classify other prevalent uropathogenic bacteria. Furthermore, all pheno-molecular AST platforms reported to date detect either DNA or mRNA, whose relatively low copy numbers (˜1-10 copies per gene or transcript (39-41)) within bacterial cells necessitate amplified detection via NAATs, and therefore must be preceded with labor-intensive nucleic acid extraction. Thus, a rapid pheno-molecular AST platform that can broaden the pathogen ID capacity, obviate NAATs, and minimize sample preparation is desirable.

Ribosomal RNA (rRNA), particularly 16S rRNA, presents a promising alternative to DNA and mRNA as a molecular surrogate of phenotypic bacterial response to antibiotics in pheno-molecular AST. Not only has 16S rRNA been well-established for phylogenetic classification of bacteria (42-44), its abundance has been shown to correlate with bacterial growth (45-47), which suggests potential compatibility with AST. Indeed, the feasibility of 16S rRNA-based pheno-molecular AST has been explored, albeit relying on either cumbersome nucleic acid amplification (37) or time-consuming culture and clinical isolation (48, 49).

Accordingly, there is a need for additional methods, and related aspects, for pathogen identification (ID) and antimicrobial susceptibility testing (AST).

### SUMMARY

The present disclosure relates, in certain aspects, to methods of identifying and determining antimicrobial susceptibility of bacteria in samples. In some embodiments, single-cell measurements of bacterial 16S rRNA are leveraged to achieve simultaneous molecular detection of bacteria and phenotypic assessment of antimicrobial susceptibility. In certain of these embodiments, a sample-to-answer is provided in about 30 minutes or less. The abundance of 16S rRNA (˜10³-10⁵ copies in a bacterial cell) makes it a suitable target for single-cell detection technology based on hybridization with sequence-specific probes in microfluidic droplets as described herein. Such detection strategies for 16S rRNA-based pheno-molecular antimicrobial susceptibility testing (AST) can also be readily multiplexed to achieve even broader pathogen analysis. Typically, the analytical strategies disclosed herein eliminate nucleic acid amplification and extraction steps, thus minimizing sample preparation and enabling sample-to-answer operation across an array of sample types. These and other aspects will be apparent upon a complete review of the present disclosure, including the accompanying figures.

In one aspect, the present disclosure provides a method of identifying and determining an antimicrobial susceptibility of bacteria in a sample. The method includes forming at least one set of droplets that each comprise at least one bacterial cell from the sample, at least one antimicrobial agent, and at least one set of probes. The set of probes comprises a plurality of probes that each comprise at least one nucleobase sequence that is at least partially complementary to at least one region of at least one 16S ribosomal RNA (rRNA) sequence of 16S rRNA from the bacterial cell and wherein the plurality of probes each comprise at least one reporter moiety and at least one quencher moiety that substantially quenches the reporter moiety at least when the plurality of probes are not hybridized to the 16S rRNA. The method also includes incubating the set of droplets for a first time period under conditions sufficient for the bacterial cells in the droplets to multiply and/or to at least produce additional 16S rRNA if the bacterial cells are not susceptible to the antimicrobial agent, or to not substantially multiply and/or to not substantially produce additional 16S rRNA if the bacterial cells are susceptible to the antimicrobial agent. The method also includes incubating the set of droplets for a second time period under conditions sufficient for one or more of the plurality of probes to hybridize with the 16S rRNA from the bacterial cells (e.g., in embodiments that involve direct detection via hybridization), and optionally, to artificially amplify the 16S rRNA from the bacterial cells (e.g., in embodiments that involve reverse transcriptase-polymerase chain reaction (RT-PCR) or other artificial nucleic acid amplification techniques). The method also includes detecting a detectable signal from the reporter moiety of one or more of the probes in one or more of the droplets, thereby identifying the bacteria. In addition, the method also includes comparing a strength of the detectable signal from the reporter moiety with a strength of a reference detectable signal, wherein when the strength of the detectable signal from the reporter moiety is greater than the strength of the reference detectable signal, the bacterial cell is indicated not to be susceptible to the antimicrobial agent, and wherein when the strength of the detectable signal from the reporter moiety is less than the strength of the reference detectable signal, the bacterial cell is indicated to be susceptible to the antimicrobial agent, thereby determining the antimicrobial susceptibility of bacteria in the sample.

In another aspect, the present disclosure provides a method of determining a presence or absence of bacteria in a sample in an absence of artificially amplifying nucleic acids, if present, in or from the sample. The method includes forming at least one set of droplets that each comprise at least an aliquot of the sample and at least one set of probes. The set of probes comprises a plurality of probes that each comprise at least one nucleobase sequence that is at least partially complementary to at least one region of at least one bacterial ribosomal RNA (rRNA) sequence. The plurality of probes each comprise at least one reporter moiety and at least one quencher moiety that substantially quenches the reporter moiety when the plurality of probes are not hybridized to bacterial rRNA. The method also includes incubating the set of droplets under conditions sufficient for one or more of the plurality of probes to hybridize with the bacterial rRNA, if present, in the sample. In addition, the method also includes determining whether a detectable signal is detected from the reporter moiety of any of the plurality of probes in any of the droplets, in which a presence of a detected detectable signal indicates that the bacteria is present in the sample, and in which an absence of a detected detectable signal indicates that the bacteria is absent from the sample.

In another aspect, the present disclosure provides a method of identifying bacteria in a sample in an absence of artificially amplifying nucleic acids in or from the sample. The method includes forming at least one set of droplets that each comprise at least one bacterial cell from the sample and at least one set of probes in which the set of probes comprises a plurality of probes that each comprise at least one nucleobase sequence that is at least partially complementary to at least one region of at least one ribosomal RNA (rRNA) sequence of rRNA from the bacterial cell and in which the plurality of probes each comprise at least one reporter moiety and at least one quencher moiety that substantially quenches the reporter moiety when the plurality of probes are not hybridized to the rRNA. The method also includes incubating the set of droplets under conditions sufficient for one or more of the plurality of probes to hybridize with the rRNA from the bacterial cells. In addition, the method also includes detecting a detectable signal from the reporter moiety of one or more of the plurality of probes in one or more of the droplets, thereby identifying the bacteria in the sample in the absence of artificially amplifying nucleic acids in or from the sample.

In another aspect, the present disclosure provides a method of identifying and determining an antimicrobial susceptibility of bacteria in a sample in an absence of artificially amplifying nucleic acids in or from the sample. The method includes forming at least one set of droplets that each comprise at least one bacterial cell from the sample, at least one antimicrobial agent, and at least one set of probes in which the set of probes comprises a plurality of probes that each comprise at least one nucleobase sequence that is at least partially complementary to at least one region of at least one ribosomal RNA (rRNA) sequence of rRNA from the bacterial cell and in which the plurality of probes each comprise at least one reporter moiety and at least one quencher moiety that substantially quenches the reporter moiety at least when the plurality of probes are not hybridized to the rRNA. The method also includes incubating the set of droplets for a first time period under conditions sufficient for the bacterial cells in the droplets to multiply and/or to at least produce additional rRNA if the bacterial cells are not susceptible to the antimicrobial agent, or to not substantially multiply and/or to not substantially produce additional rRNA if the bacterial cells are susceptible to the antimicrobial agent, and incubating the set of droplets for a second time period under conditions sufficient for one or more of the plurality of probes to hybridize with the rRNA from the bacterial cells. The method also includes incubating the set of droplets for a second time period under conditions sufficient for the plurality of probes to hybridize with the rRNA from the bacterial cells. The method also includes detecting a detectable signal from the reporter moiety of one or more of the probes in one or more of the droplets, thereby identifying the bacteria, and comparing a strength of the detectable signal from the reporter moiety with a strength of a reference detectable signal, wherein when the strength of the detectable signal from the reporter moiety is greater than the strength of the reference detectable signal, the bacterial cell is indicated not to be susceptible to the antimicrobial agent, and wherein when the strength of the detectable signal from the reporter moiety is less than the strength of the reference detectable signal, the bacterial cell is indicated to be susceptible to the antimicrobial agent, thereby determining the antimicrobial susceptibility of bacteria in the absence of artificially amplifying nucleic acids in or from the sample.

In another aspect, the present disclosure provides a method of identifying bacteria in a sample in an absence of artificially amplifying nucleic acids in or from the sample. The method includes forming at least one set of droplets that each comprise at least one bacterial cell from the sample and at least one set of probes selected from Table 1, and incubating the set of droplets under conditions sufficient for one or more of the probes to hybridize with 16S ribosomal RNA (rRNA) from the bacterial cells. The method also includes detecting a detectable signal from the reporter moiety of one or more of the probes in one or more of the droplets, thereby identifying the bacteria in the sample in the absence of artificially amplifying nucleic acids in or from the sample.

In another aspect, the present disclosure provides a method of identifying and determining an antimicrobial susceptibility of bacteria in a sample in an absence of artificially amplifying nucleic acids in or from the sample. The method includes forming at least one set of droplets that each comprise at least one bacterial cell from the sample, at least one antimicrobial agent, and at least one set of probes selected from Table 1, and incubating the set of droplets for a first time period under conditions sufficient for the bacterial cells in the droplets to multiply and/or to at least produce additional 16S ribosomal RNA (rRNA) if the bacterial cells are not susceptible to the antimicrobial agent, or to not substantially multiply and/or to not substantially produce additional 16S rRNA if the bacterial cells are susceptible to the antimicrobial agent. The method also includes incubating the set of droplets for a second time period under conditions sufficient for one or more of the probes to hybridize with the 16S rRNA from the bacterial cells, and detecting a detectable signal from the reporter moiety of one or more of the probes in one or more of the droplets, thereby identifying the bacteria. In addition, the method also includes comparing a strength of the detectable signal from the reporter moiety with a strength of a reference detectable signal, wherein when the strength of the detectable signal from the reporter moiety is greater than the strength of the reference detectable signal, the bacterial cell is indicated not to be susceptible to the antimicrobial agent, and wherein when the strength of the detectable signal from the reporter moiety is less than the strength of the reference detectable signal, the bacterial cell is indicated to be susceptible to the antimicrobial agent, thereby determining the antimicrobial susceptibility of bacteria in the absence of artificially amplifying nucleic acids in or from the sample.

In some embodiments, the sample comprises a urine sample. In some embodiments, the methods disclosed herein include quantifying an amount of the bacteria in the sample. In some embodiments, the methods disclosed herein include obtaining the sample from at least one subject. In some embodiments, the subject is a human subject. In some embodiments, the methods disclosed herein include administering one or more antimicrobial therapeutic agents to the subject upon identifying and determining the antimicrobial susceptibility of the bacteria in the sample. In some embodiments, the plurality of droplets each comprise a single bacterial cell from the sample. In some embodiments, the antimicrobial agent comprises an antimicrobial therapeutic agent. In some embodiments, the antimicrobial agent is selected from the group selected from: a carbapenem, a fluoroquinolone, a β-lactam, a aminoglycoside, trimethoprim/sulfmethoxazole, and the like. In some embodiments, the rRNA sequence comprises a 16S rRNA sequence. Typically, the methods disclosed herein include forming the droplets in a microfluidic device.

In some embodiments, the set of probes comprises a plurality of hybridization probes. In some embodiments, the set of probes comprises a plurality of hairpin probes. In some embodiments, a given probe comprises a first strand that comprises a peptide nucleic acid (PNA) molecule that comprises the nucleobase sequence that is at least partially complementary to the at least one region of the at least one bacterial rRNA sequence, and a second strand that comprises a nucleic acid molecule, wherein the second strand comprises a nucleobase sequence that is sufficiently complementary to a nucleobase sequence of the first strand such that the first and second strands hybridize with one another under conditions that are not sufficient for the probes to hybridize with the bacterial rRNA. In some embodiments, the first strand comprises a sequence length of about 15 to about 17 nucleobases and the second strand comprises a sequence length of about 10 to about 12 nucleobases. In some embodiments, the first strand comprises the reporter moiety and the second strand comprises the quencher moiety. In some embodiments, the first strand comprises the quencher moiety and the second strand comprises the reporter moiety. In some embodiments, the set of probes is selected from Table 1. In some embodiments, the at least one set of probes comprises at least first and second sets of probes that comprise reporter moieties that differ from one another. In some embodiments, the probes comprise one or more modified nucleobases. In some embodiments, the reporter moiety comprises at least one fluorophore.

In some embodiments, the reference detectable signal is obtained from droplets that comprise the bacterial cell and the set of probes and lack the antimicrobial agent. In some embodiments, a given droplet comprises a volume of about 10 picoliters (pL) or less. In some embodiments, a given droplet comprises between about 10³ and about 10⁵ copies of the rRNA. In some embodiments, the first time period comprises about 15 minutes or less. In some embodiments, the methods disclosed herein include incubating the set of droplets for the first time period at a temperature of about 37° C. In some embodiments, the second time period comprises about 20 minutes or less. In some embodiments, the methods disclosed herein include incubating the set of droplets for the second time period at a temperature of about 60° C. In some embodiments, the methods disclosed herein include lysing the bacterial cells in the droplets. In some embodiments, the methods disclosed herein include incubating the set of droplets for about two minutes at a temperature of about 95° C. to lyse the bacterial cells in the droplets.

In some embodiments, the bacterial cell comprises a Gram-negative bacterial cell. In some embodiments, the bacterial cell comprises an E. coli cell, a P. mirabilis cell, a K. pneumoniae cell, or a P. aeruginosa cell. In some embodiments, the bacterial cell is from the Enterobacterales order. In some embodiments, the bacterial cell is from the eubacterial kingdom.

In another aspect, the present disclosure provides a reaction mixture, comprising at least one set of probes selected from Table 1. In some embodiments, the reaction mixture disclosed herein further includes at least one antimicrobial agent. In some embodiments, the reaction mixture disclosed herein further includes at least one bacterial cell.

In another aspect, the present disclosure provides a device that includes a body structure that defines at least one droplet formation cavity, at least one fluidic channel, and at least one detectable signal detection cavity. The droplet formation cavity and the detectable signal detection cavity fluidly communicate with one another via the fluidic channel. The droplet formation cavity fluidly communicates, or is capable of fluidly communicating, with at least one sample source, at least one reagent source, and at least one immiscible fluid source (e.g., an oil reservoir or the like). The reagent source comprises at least one set of probes in which the set of probes comprises a plurality of probes that each comprise at least one nucleobase sequence that is at least partially complementary to at least one region of at least one ribosomal RNA (rRNA) sequence of rRNA from at least one bacterial cell and in which the plurality of probes each comprise at least one reporter moiety and at least one quencher moiety that substantially quenches the reporter moiety at least when the plurality of probes are not hybridized to the rRNA. The droplet formation cavity is configured to form droplets that comprise at least some of the set of probes from the reagent source and at least an aliquot of a sample from the sample source. The fluidic channel comprises at least one droplet culture region, at least one bacterial cell lysis region, and at least one hybridization region, wherein the droplet culture region is configured to thermally communicate with a droplet culture thermal modulator, wherein the bacterial cell lysis region is configured to thermally communicate with a lysis thermal modulator, and wherein the hybridization region is configured to thermally communicate with a hybridization thermal modulator. The detectable signal detection cavity is configured to detectably communicate with one or more detectors that are capable of detecting detectable signals from reporter moieties of the probes when the probes hybridize with the rRNA from the at least one bacterial cell. The device is not configured to artificially amplify nucleic acids, if present, in or from samples or the aliquot of the sample from the sample source. In some embodiments, the set of probes is selected from Table 1. In some embodiments, a kit includes the device.

In another aspect, the present disclosure provides a system that includes a device, comprising a body structure that defines at least one droplet formation cavity, at least one fluidic channel, and at least one detectable signal detection cavity. The droplet formation cavity and the detectable signal detection cavity fluidly communicate with one another via the fluidic channel. The droplet formation cavity fluidly communicates, or is capable of fluidly communicating, with at least one sample source, at least one reagent source, and at least one immiscible fluid source, which reagent source comprises at least one set of probes, wherein the set of probes comprises a plurality of probes that each comprise at least one nucleobase sequence that is at least partially complementary to at least one region of at least one ribosomal RNA (rRNA) sequence of rRNA from at least one bacterial cell and wherein the plurality of probes each comprise at least one reporter moiety and at least one quencher moiety that substantially quenches the reporter moiety at least when the plurality of probes are not hybridized to the rRNA, which droplet formation cavity is configured to form droplets that comprise at least some of the set of probes from the reagent source and at least an aliquot of a sample from the sample source. The fluidic channel comprises at least one droplet culture region, at least one bacterial cell lysis region, and at least one hybridization region, wherein the droplet culture region is configured to thermally communicate with a droplet culture thermal modulator, wherein the bacterial cell lysis region is configured to thermally communicate with a lysis thermal modulator, and wherein the hybridization region is configured to thermally communicate with a hybridization thermal modulator. The detectable signal detection cavity is configured to detectably communicate with one or more detectors that are capable of detecting detectable signals from reporter moieties of the probes when the probes hybridize with the rRNA from the at least one bacterial cell. The device is not configured to artificially amplify nucleic acids, if present, in or from samples or the aliquot of the sample from the sample source. The system also includes a droplet culture thermal modulator, a lysis thermal modulator, and a hybridization thermal modulator. The system also includes a detector that is capable of detecting detectable signals from reporter moieties of the probes when the probes hybridize with the rRNA from the at least one bacterial cell. In addition, the system also includes a controller operably connected to the droplet culture thermal modulator, the lysis thermal modulator, the hybridization thermal modulator, and the detector, which controller is configured to modulate temperatures of the droplet culture thermal modulator, the lysis thermal modulator, and the hybridization thermal modulator, and to effect detection of the detectable signals from the reporter moieties via the detector.

### BRIEF DESCRIPTION OF THE DRAWINGS

The accompanying drawings, which are incorporated in and constitute a part of this specification, illustrate certain embodiments, and together with the written description, serve to explain certain principles of the methods, devices, kits, systems, and related computer readable media disclosed herein. The description provided herein is better understood when read in conjunction with the accompanying drawings which are included by way of example and not by way of limitation. It will be understood that like reference numerals identify like components throughout the drawings, unless the context indicates otherwise. It will also be understood that some or all of the figures may be schematic representations for purposes of illustration and do not necessarily depict the actual relative sizes or locations of the elements shown.

FIG. 1 is a flow chart that schematically shows exemplary method steps of determining a presence or absence of bacteria in a sample according to some aspects disclosed herein.

FIG. 2 is a flow chart that schematically shows exemplary method steps of identifying bacteria in a sample according to some aspects disclosed herein.

FIG. 3 is a flow chart that schematically shows exemplary method steps of identifying and determining an antimicrobial susceptibility of bacteria in a sample according to some aspects disclosed herein.

FIG. 4 is a schematic diagram of an exemplary system suitable for use with certain aspects disclosed herein.

FIG. 5. DropDx platform for rapid ID and AST of urinary tract infections. (A) We have developed a urine sample-to-answer platform that leverages droplet microfluidics for single-cell detection of bacterial 16S rRNA in order to rule out or confirm bacterial infection, identify the causative uropathogen, and assess its antimicrobial resistance, all within 30 min of operation. (B) Bacteria in urine samples are digitized into picoliter droplets along with 16S rRNA-specific fluorogenic PNA probes and/or antibiotics before being subjected to on-chip culture/antibiotic exposure for 10 min and probe hybridization assay for 16 min. Droplets are individually interrogated by a 2-color laser induced fluorescence (LIF) detector, and (C) the fluorescence color and intensity of droplets are used to detect the presence of specific 16S rRNA sequences for identifying uropathogenic bacteria. The difference in probe fluorescence intensities from antibiotic-dosed and antibiotic-free droplets are used to determine the relative production of 16S rRNA in single cells, which can be used to assess the antimicrobial resistance of the pathogen.

FIG. 6. Two-color laser induced fluorescence (LIF) detector and bulk reaction assessment device. (A) The LIF detector used for both bulk and droplet fluorescence measurements consists of 2 distinct laser illumination sources (488 nm and 552 nm) and 2 avalanche photodiode sensors (APDs 1 & 2). (B) Fluorescence signal for all bulk reactions (used for assay validation and optimization) were measured in custom microfluidic devices that contained parallel channels with 10-μm detection constrictions.

FIG. 7. Modular droplet device for flexible assay characterization. A PDMS microfluidic device consisting of a droplet generation region and a normally disconnected droplet detection region served as the platform for assay characterization in droplets. Generated droplets enter into a Tygon tube which traverses over heaters that facilitate bacterial lysis (at 95° C.) and PNA probe hybridization (at 60° C.) before re-entering the device for detection. Hybridization duration of droplets was controlled by varying the length of Tygon tubing that rested on the hybridization heater. Droplet volume was controlled by controlling the height of the channels within in the device. As such, separate devices were used for generation of 1 pL, 4 pL, and 30 pL droplet volumes. Scale bars are ˜100 μm.

FIG. 8. One-step sample pretreatment protocol. (A) The one-step pretreatment protocol includes a single infusion of urine/MH solution through an appropriately sized syringe filter. (i) Some urine samples can emit a high auto-fluorescence background. In such cases, (ii) 2-fold or (iii) or 4-fold dilution of the sample in MH broth is necessary to improve signal to background ratio of positive droplets versus empty droplets, and recover the expected frequency of positive droplets based on the input concentration of bacteria (˜10%, here). (B) Particulates in urine can impose a high limit of blank (i.e., frequency of empty droplets with high fluorescence intensities from blank/culture-negative urine samples) and hamper bacteria quantification. Three different syringe filters of varying pore sizes—GE Whatman (pore size 1.2 μm), GE Whatman (pore size: 2.0 μm), and iPOC-Dx Primecare (pore size gradient from 35 μm to 2.5 μm) were tested. One-step filtration of the urine/MH mixture through the iPOC-Dx filter reduces limit of blank by more than an order of magnitude, while ensuring up to 94% bacterial recovery for quantification.

FIG. 9. PNA probe hybridization assay for specific detection of uropathogens. (A) PNA probes were designed specific to the (i) E. coli and (ii) P. mirabilis species, the (iii) Enterobacterales order, and the (iv) eubacterial kingdom, in order to be able to detect all predominant uropathogens. We ensure that the designed probes can detect signal from target bacteria over blank urine background (N) by measuring probe fluorescence in the presence of E. coli (EC), P. mirabilis (PM), K. pneumoniae (KP), and P. aeruginosa (PA) (*p<0.05, **p<0.01, ***p<0.001, no asterisks between bars indicates no significant difference). (B) The assay works across a wide range of (i) lysis temperatures and (ii) hybridization temperatures (green: selected temperatures). (C) Bulk-based pheno-molecular AST of reference E. coli ATCC 25922 and multi-drug resistant E. coli BAA 2471 using hybridization detection of 16S rRNA is feasible, but needs >90 min of culture/antibiotic exposure to differentiate the effect of gentamicin on the susceptible and the resistant strains of E. coli. All error bars represent 1 standard deviation.

FIG. 10. Bulk pheno-molecular AST of E. coli 25922. PNA probe signal from E. coli cells increases for increasing culture durations, indicative of increased 16S rRNA production as bacteria replicate. In the presence of gentamicin, there is a relatively lower production of 16S rRNA over increasing incubation durations. In bulk, the inhibitory effect of 16S rRNA is noticeable after 90 min of culture.

FIG. 11. Single-cell detection of bacterial 16S rRNA from urine using microfluidic droplets. (A) (i) Urine samples of distinctly different color and turbidity can be discretized using flow-focusing to generate monodisperse droplets (scale bars 50 μm) of (ii) 4±1 pL volume. (B) Droplet fluorescence peak traces (i) Without E. coli, droplets emit baseline fluorescence signal, and have a positive droplet rate of 0.0079% (also known as the average limit of blank). (ii) In the presence of E. coli, droplets emit a higher fluorescence signal, and have a positive droplet rate of 6.67%. (C) Droplet-based quantification of E. coli in urine across 4 orders of magnitude within the clinically relevant dynamic range for UTIs (10⁴ to 2×10⁷ CFU/mL), R²=0.992. (D)(i) Reduction in droplet volume from 30 pL to 4 pL to 1 pL results in lower background fluorescence signals (scale bars 50 μm, R²=0.904). (ii) Compared to larger 30 pL droplets, 4 pL droplets facilitate faster generation of differentiable fluorescence signal over the reduced local background, as quickly as within 15 min. All error bars represent 1 standard deviation.

FIG. 12. Bulk sensitivity of the PNA probe assay. The signal from bacteria spiked into urine samples were measured and compared to the fluorescence from no-bacteria controls in the same samples. For bulk reactions, at least 1.5×10⁸ cfu/mL bacteria should be present in order to effectively measure signal over urine background.

FIG. 13. Accelerating antimicrobial susceptibility assessment via quantitative measurement of 16S rRNA from single cells. (A) LIF detection of droplets containing E. coli cells suspended in MH broth (i) without 30 min culture results in the expected 8% positive droplet frequency (7.02% observed), (ii) following 30 min culture results in higher positive droplet intensities (indicative of higher 16S rRNA production) and 7.70% frequency, and (iii) and after 30 min culture along with bactericidal gentamicin results in lower positive droplet intensities (indicative of relatively lower 16S rRNA production) and 3.12% frequency. (B) Resistant E. coli can be differentiated from reference E. coli spiked into urine by comparing the positive droplet percentage from cells subject to antibiotic and no-antibiotic conditions (“Normalized Positive Droplet Population”) for culture/drug exposure durations as low as 10 min. (C) Resistant and susceptible strains of E. coli can be differentiated using our platform for 3 different antibiotics spanning distinct classes—gentamicin (aminoglycoside), ciprofloxacin (fluoroquinolone), and ampicillin (beta lactam). Error bars represent 1 standard deviation.

FIG. 14. Quantification of antibiotic effect on E. coli in droplets. For E. coli suspended in MH broth exposed to gentamicin, the normalized positive droplet population decreases as the concentration of gentamicin increases.

FIG. 15. Schematic of the entire DropDx platform (not to scale). The experimental setup consists of a modular (not pictured here) or integrated microfluidic device that rests on individually controlled Peltier heaters. The detection region of the device is aligned to a 2-color LIF detector. Syringe pumps are used to control the flow rates of urine samples, PNA probes, and droplet generation oil, and are finely tuned to generate stable droplets and propel the droplets through the device for the required incubation durations.

FIG. 16. Thermal platform characterization. (A) While testing clinical samples, the DropDx device was affixed to a 3-temperature heating rig via thermally conductive paste. (B) The thermal rig is able to reliably deliver the correct temperature to the rig with minimal spatial and temporal temperature variation.

FIG. 17. DropDx device and droplet residence time. The residence time of droplets traversing each zone in the droplet device was measured by tracking the front of generated droplets. Droplets spend (i) approximately 10 min in the culture/drug exposure zone, (ii) approximately 2 min in the lysis zone, and (iii) approximately 14 min in the hybridization zone.

FIG. 18. DropDx clinical comparison study of 50 deidentified patient samples from Johns Hopkins Hospital. (A) Each sample was simultaneously tested using clinical standard ID/AST tests as well as with 2 DropDx devices for measurements without and with ciprofloxacin. For ID, we used a combination of EC, EB, and UNI probes. (B) Our 7-outcome DropDx workflow is used to determine if there is bacterial infection present, whether the infecting pathogen is E. coli, whether the infecting pathogen is in the Enterobacterales order, or whether the infecting pathogen is a different (Gram-negative) bacteria and to assess the susceptibility of the infecting pathogen to ciprofloxacin. (C) Unbiased thresholding for each diagnostic metric was conducted in pilot studies using ROC curve analysis, and the final data groups and resulting ROC curves are plotted for (i) differentiating culture-positive from culture-negative samples (AUC: 0.964), for (ii) differentiating E. coli from non-E. coli samples (AUC: 1.000), for (iii) differentiating Enterobacterales from non-Enterobacterales samples (AUC: 0.963), and for (D) differentiating ciprofloxacin resistant from susceptible samples (AUC: 0.951). Importantly, DropDx has a categorical agreement of 95.3% with no major errors. Error bars represent mean and standard error. P-values calculated from unpaired one-tailed t-tests.

FIG. 19. Validation of pheno-molecular AST in the clinical comparison study. Of the 43 culture-positive samples tested, DropDx can accurately differentiate ciprofloxacin-resistant from ciprofloxacin-susceptible samples (p=0.02), resulting in a 95.3% categorical agreement with the clinical standard methodology.

FIG. 20. Clinical comparison study workflow. Data collection was divided into 3 phases. Phase 1, “AST Pilot Study” included the first 15 urine samples tested. Pilot studies were used to establish a data-agnostic threshold for susceptibility/resistance calls and determine the minimum number of samples required for adequate statistical power. Phase 2, “ID Validation Study” included the next 16 samples tested and was used to set up thresholds for the ID classification categories used in this workflow. Phase 2 was also used to validate our measurements for susceptibility/resistance. The final set of samples was used to validate both ID classification categories as well as susceptibility/resistance.

FIG. 21. Pilot studies for power analysis and threshold determination. (A) Data collected in Phase 1, the AST Pilot Study, was plotted for each category of sample interrogated. Ciprofloxacin-resistant samples (represented by inverted triangles) are well separated from ciprofloxacin-susceptible samples (represented by circles), with 1 false-resistant call. (B) (i) Power analysis was conducted using the pilot study data to determine that at least 20 samples must be interrogated in the validation phase to ensure a statistical power (1-β) of 90% and a confidence (α) of 95%. (ii) ROC analysis of the pilot study data was used to determine a data-agnostic threshold that maximizes the Youden's Index of the pilot dataset. This threshold was kept constant for all subsequent data analyzed. (C) Power analyses and ROC-based threshold determination was repeated in Phase 2, the ID Pilot Study, for each classification criteria utilized in DropDx.

### DEFINITIONS

In order for the present disclosure to be more readily understood, certain terms are first defined below. Additional definitions for the following terms and other terms may be set forth throughout the specification. If a definition of a term set forth below is inconsistent with a definition in an application or patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

It is also to be understood that the terminology used herein is for the purpose of describing particular embodiments only and is not intended to be limiting. Further, unless defined otherwise, all technical and scientific terms used herein have the same meaning as commonly understood by one of ordinary skill in the art to which this disclosure pertains. In describing and claiming the methods, microfluidic devices or systems, and component parts, the following terminology, and grammatical variants thereof, will be used in accordance with the definitions set forth below.

About: As used herein, “about” or “approximately” or “substantially” as applied to one or more values or elements of interest, refers to a value or element that is similar to a stated reference value or element. In certain embodiments, the term “about” or “approximately” or “substantially” refers to a range of values or elements that falls within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value or element unless otherwise stated or otherwise evident from the context (except where such number would exceed 100% of a possible value or element).

Administering: As used herein, the terms “administering” and “administration” refer to any method of providing a pharmaceutical preparation or other treatment to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

Artificially Amplifying: As used herein, “artificially amplifying” in the context of nucleic acids refers to the non-natural production of multiple copies of a polynucleotide, or a portion of the polynucleotide, typically starting from a small amount of the polynucleotide (e.g., a single polynucleotide molecule), where the amplification products or amplicons are generally detectable. Amplification of polynucleotides encompasses a variety of chemical and enzymatic processes. The generation of multiple DNA copies from one or a few copies of a target or template DNA molecule during a polymerase chain reaction (PCR) or a ligase chain reaction (LCR) are forms of amplification. Amplification is not limited to the strict duplication of the starting molecule. For example, the generation of multiple cDNA molecules from a limited amount of RNA in a sample using RT-PCR is a form of amplification. Furthermore, the generation of multiple RNA molecules from a single DNA molecule during the process of transcription is also a form of amplification.

Communicate: As used herein, “communicate” refers to the direct or indirect transfer or transmission, and/or capability of directly or indirectly transferring or transmitting, something at least from one thing to another thing. In some embodiments, for example, device channels and/or cavities fluidly communicate between or among one another when fluidic materials can flow between or among those channels and/or cavities. To further illustrate, device or system components thermally communicate with one another when thermal energy can be transferred between those components.

Detect: As used herein, “detect,” “detecting,” or “detection” refers to an act of determining the existence or presence of one or more target biomolecules (e.g., nucleic acids, proteins, etc.) in a sample.

Detectable Signal: As used herein, “detectable signal” refers to signal output at an intensity or power sufficient to be detected in a given detection system. In certain embodiments, a detectable signal is emitted from a label (e.g., a fluorescent label or the like) associated with a given probe nucleic acid.

Hairpin Probe: As used herein, “hairpin probe” refers to an oligonucleotide that can be used to effect target nucleic acid detection and that includes at least one region of self-complementarity such that the probe is capable of forming a hairpin or loop structure under selected conditions. Typically, hairpin probes include one or more labeling moieties. In one exemplary embodiment, quencher moieties and reporter moieties are positioned relative to one another in the hairpin probes such that the quencher moieties at least partially quench light emissions from the reporter moieties when the probes are in hairpin confirmations. In contrast, when the probes in these embodiments are not in hairpin confirmations (e.g., when the probes are hybridized with target nucleic acids), light emissions the acceptor reporter moieties are generally detectable. Hairpin probes are also known as molecular beacons in some of these embodiments. Hairpin probes can also function as 5′-nuclease probes or hybridization probes in certain embodiments.

Hybridization Probe: As used herein, “hybridization probe” refers an oligonucleotide that includes at least one labeling moiety that can be used to effect target nucleic acid detection. In some embodiments, hybridization probes function in pairs. In some of these embodiments, for example, a first hybridization probe of a pair includes at least one donor moiety at or proximal to its 3′-end, while the second hybridization probe of the pair includes at least one acceptor moiety (e.g., LC-Red 610, LC-Red 640, LC-Red 670, LC-Red 705, JA-270, CY5, or CY5.5) at or proximal to its 5′-end.

Hybridize: As used herein, “hybridize” refers to the formation of a double-stranded molecule between sufficiently complementary polynucleotides, or analogs or modified forms thereof (e.g., nucleic acids hybridized with PNAs or the like).

Label: As used herein, “label” refers to a moiety attached (covalently or non-covalently), or capable of being attached, to a molecule, which moiety provides or is capable of providing information about the molecule (e.g., descriptive, identifying, etc. information about the molecule). Exemplary labels include donor moieties, acceptor moieties, fluorescent labels, non-fluorescent labels, calorimetric labels, chemiluminescent labels, bioluminescent labels, radioactive labels, mass-modifying groups, antibodies, antigens, biotin, haptens, and enzymes (including, e.g., peroxidase, phosphatase, etc.).

Mixture: As used herein, “mixture” refers to a combination of two or more different components.

Probe Nucleic Acid: As used herein, “probe nucleic acid” or “probe” refers to a labeled or unlabeled oligonucleotide capable of selectively hybridizing to a target or template nucleic acid under suitable conditions. Typically, a probe is sufficiently complementary to a specific target sequence contained in a nucleic acid sample to form a stable hybridization duplex with the target sequence under a selected hybridization condition, such as, but not limited to, a stringent hybridization condition. A hybridization assay carried out using a probe under sufficiently stringent hybridization conditions permits the selective detection of a specific target sequence. The term “hybridizing region” refers to that region of a nucleic acid that is exactly or substantially complementary to, and therefore capable of hybridizing to, the target sequence. For use in a hybridization assay for the discrimination of single nucleotide differences in sequence, the hybridizing region is typically from about 8 to about 100 nucleotides in length. Although the hybridizing region generally refers to the entire oligonucleotide, the probe may include additional nucleotide sequences that function, for example, as linker binding sites to provide a site for attaching the probe sequence to a solid support. A probe of the invention is generally included in a nucleic acid that comprises one or more labels (e.g., donor moieties, acceptor moieties, and/or quencher moieties), such as exonuclease probe (e.g., a 5′-nuclease probe), a hybridization probe, a fluorescent resonance energy transfer (FRET) probe, a hairpin probe, or a molecular beacon, which can also be utilized to detect hybridization between the probe and target nucleic acids in a sample. In some embodiments, the hybridizing region of the probe is completely complementary to the target sequence. However, in general, complete complementarity is not necessary (i.e., nucleic acids can be partially complementary to one another); stable hybridization complexes may contain mismatched bases or unmatched bases. Modification of the stringent conditions may be necessary to permit a stable hybridization complex with one or more base pair mismatches or unmatched bases. Stability of the target/probe hybridization complex depends on a number of variables including length of the oligonucleotide, base composition and sequence of the oligonucleotide, temperature, and ionic conditions. One of skill in the art will recognize that, in general, the exact complement of a given probe is similarly useful as a probe.

Nucleic Acid: As used herein, “nucleic acid” refers to a naturally occurring or synthetic oligonucleotide or polynucleotide, whether DNA or RNA or DNA-RNA hybrid, single-stranded or double-stranded, sense or antisense, which is capable of hybridization to a complementary nucleic acid by Watson-Crick base-pairing. Nucleic acids can also include nucleotide analogs (e.g., bromodeoxyuridine (BrdU)), and non-phosphodiester internucleoside linkages (e.g., peptide nucleic acid (PNA) or thiodiester linkages). In particular, nucleic acids can include, without limitation, DNA, RNA, cDNA, gDNA, ssDNA, dsDNA, cfDNA, ctDNA, or any combination thereof.

Reaction Mixture: As used herein, “reaction mixture” refers a mixture that comprises molecules that can participate in and/or facilitate a given reaction or assay. To illustrate, in some embodiments, a reaction mixture includes a solution containing reagents necessary to carry out a nucleic acid hybridization reaction.

Sample: As used herein, “sample” means anything capable of being analyzed by the methods, cartridges and/or devices disclosed herein. Samples can include a tissue or organ from a subject; a cell (either within a subject, taken directly from a subject, or a cell maintained in culture or from a cultured cell line); a ceil lysate (or lysate fraction) or cell extract; or a solution containing one or more biomolecules derived from a cell or cellular material (e.g., a nucleic acid, a protein, etc.), which is assayed as described herein. A sample may also be any body fluid or excretion (for example, but not limited to, blood, urine, stool, saliva, tears, bile) that contains cells, cell components, or non-cellular fractions. Additional examples of samples include environment and forensic samples. Samples can also include infectious disease agents (e.g., bacteria, viruses, etc.) or plant matter, among other sample types.

Subject: As used herein, “subject” refers to an animal, such as a mammalian species (e.g., human) or avian (e.g., bird) species, or other organism, such as a plant. More specifically, a subject can be a vertebrate, e.g., a mammal such as a mouse, a primate, a simian or a human. Animals include farm animals (e.g., production cattle, dairy cattle, poultry, horses, pigs, and the like), sport animals, and companion animals (e.g., pets or support animals). A subject can be a healthy individual, an individual that has or is suspected of having a disease, infection, or a predisposition to the disease, or an individual that is in need of therapy or suspected of needing therapy. The terms “individual” or “patient” are intended to be interchangeable with “subject.”

System: As used herein, “system” in the context of analytical instrumentation refers a group of objects and/or devices that form a network for performing a desired objective.

### DETAILED DESCRIPTION

Empiric broad-spectrum antimicrobial treatment of urinary tract infections (UTIs) or other infections has contributed to widespread antimicrobial resistance. Clinical adoption of specific evidence-based treatments necessitates rapid diagnostic methods that enable pathogen identification (ID) and antimicrobial susceptibility testing (AST) that can be easily operated, with minimal sample preparation. In response, the present disclosure presents, in certain embodiments, microfluidic droplet-based approaches for achieving both ID and AST from urine or other sample types within as little as 30 minutes. Some embodiments disclosed herein utilize fluorogenic peptide nucleic acid probes for hybridization detection of 16S rRNA from single bacterial cells captured in picoliter droplets, enabling molecular identification of uropathogenic bacteria directly from urine samples in as little as 16 minutes, precluding the need for nucleic acid amplification and associated sample preparation. In some embodiments, in-droplet quantitative measurements of 16S rRNA from single bacterial cells also provides an effective surrogate for phenotypic AST, after as short as 10 minutes of exposure to three common oral and parenteral UTI antibiotics. The present disclosure also provides, in some embodiments, a fully integrated device, instrument/system, and screening workflow to test urine and other samples for 1 of 7 unique diagnostic outcomes including the presence or absence of Gram-negative bacterial infection, molecular ID of the infecting pathogen as E. coli, an Enterobacterales, or other organisms, and assessment of bacterial susceptibility to ciprofloxacin, among other antimicrobial agents. In an exemplary clinical comparison study disclosed herein with 50 patient urine samples, the platform demonstrates excellent performance in both ID and AST compared to the clinical standard methods (AUCs>0.95), all within a small fraction of the turnaround time, highlighting its clinical utility.

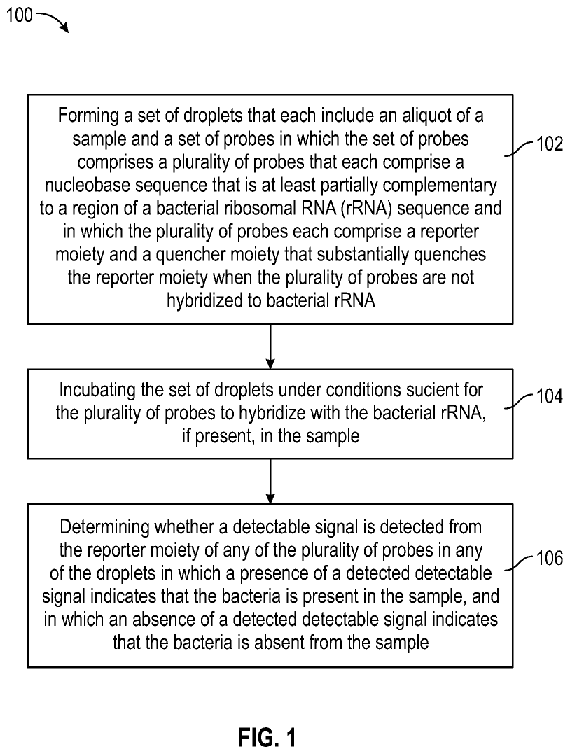

To illustrate, FIG. 1 is a flow chart that schematically shows exemplary method steps of determining a presence or absence of bacteria in a sample according to some aspects disclosed herein. As shown, method 100 includes forming at least one set of droplets that each comprise at least an aliquot of the sample and at least one set of probes (e.g., selected from Table 1) (step 102). The set of probes comprises a plurality of probes that each comprise at least one nucleobase sequence that is at least partially complementary to at least one region of at least one bacterial ribosomal RNA (rRNA) sequence. The plurality of probes each comprise at least one reporter moiety and at least one quencher moiety that substantially quenches the reporter moiety when the plurality of probes are not hybridized to bacterial rRNA. Method 100 also includes incubating the set of droplets under conditions sufficient for one or more of the plurality of probes to hybridize with the bacterial rRNA, if present, in the sample (step 104). In addition, method 100 also includes determining whether a detectable signal is detected from the reporter moiety of any of the plurality of probes in any of the droplets, in which a presence of a detected detectable signal indicates that the bacteria is present in the sample, and in which an absence of a detected detectable signal indicates that the bacteria is absent from the sample (step 106).

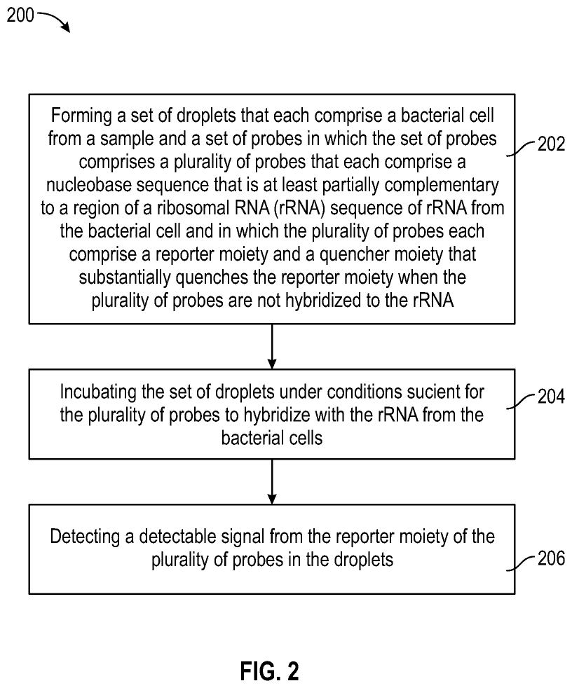

To further illustrate, FIG. 2 is a flow chart that schematically shows exemplary method steps of identifying bacteria in a sample according to some aspects disclosed herein. As shown, method 200 includes forming at least one set of droplets that each comprise at least one bacterial cell from the sample and at least one set of probes (e.g., selected from Table 1) in which the set of probes comprises a plurality of probes that each comprise at least one nucleobase sequence that is at least partially complementary to at least one region of at least one ribosomal RNA (rRNA) sequence of rRNA from the bacterial cell and in which the plurality of probes each comprise at least one reporter moiety and at least one quencher moiety that substantially quenches the reporter moiety when the plurality of probes are not hybridized to the rRNA (step 202). Method 200 also includes incubating the set of droplets under conditions sufficient for one or more of the plurality of probes to hybridize with the rRNA from the bacterial cells (step 204). In addition, method 200 also includes detecting a detectable signal from the reporter moiety of one or more of the plurality of probes in one or more of the droplets, thereby identifying the bacteria in the sample in the absence of artificially amplifying nucleic acids in or from the sample (step 206).

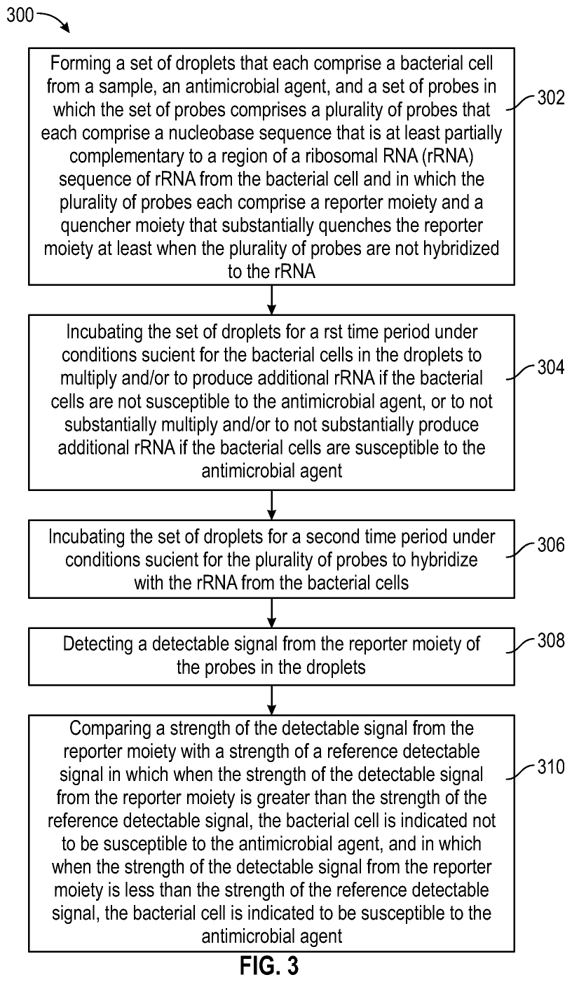

As a further illustration, FIG. 3 is a flow chart that schematically shows exemplary method steps of identifying and determining an antimicrobial susceptibility of bacteria in a sample according to some aspects disclosed herein. Method 300 includes forming at least one set of droplets that each comprise at least one bacterial cell from the sample, at least one antimicrobial agent, and at least one set of probes (e.g., selected from Table 1) in which the set of probes comprises a plurality of probes that each comprise at least one nucleobase sequence that is at least partially complementary to at least one region of at least one ribosomal RNA (rRNA) sequence of rRNA from the bacterial cell and in which the plurality of probes each comprise at least one reporter moiety and at least one quencher moiety that substantially quenches the reporter moiety at least when the plurality of probes are not hybridized to the rRNA (step 302). Method 300 also includes incubating the set of droplets for a first time period under conditions sufficient for the bacterial cells in the droplets to multiply and/or to at least produce additional rRNA if the bacterial cells are not susceptible to the antimicrobial agent, or to not substantially multiply and/or to not substantially produce additional rRNA if the bacterial cells are susceptible to the antimicrobial agent, and incubating the set of droplets for a second time period under conditions sufficient for one or more of the plurality of probes to hybridize with the rRNA from the bacterial cells (step 304). Method 300 also includes incubating the set of droplets for a second time period under conditions sufficient for the plurality of probes to hybridize with the rRNA from the bacterial cells (step 306). Method 300 also includes detecting a detectable signal from the reporter moiety of one or more of the probes in one or more of the droplets, thereby identifying the bacteria, and comparing a strength of the detectable signal from the reporter moiety with a strength of a reference detectable signal, wherein when the strength of the detectable signal from the reporter moiety is greater than the strength of the reference detectable signal, the bacterial cell is indicated not to be susceptible to the antimicrobial agent, and wherein when the strength of the detectable signal from the reporter moiety is less than the strength of the reference detectable signal, the bacterial cell is indicated to be susceptible to the antimicrobial agent, thereby determining the antimicrobial susceptibility of bacteria in the absence of artificially amplifying nucleic acids in or from the sample (steps 308 and 310).

Essentially any sample type is optionally used in performing the methods disclosed herein. In some embodiments, the sample comprises a urine sample. In some embodiments, the methods disclosed herein include quantifying an amount of the bacteria in the sample. In some embodiments, the methods disclosed herein include obtaining the sample from at least one subject. In some embodiments, the subject is a human subject. In some embodiments, the methods disclosed herein include administering one or more antimicrobial therapeutic agents to the subject upon identifying and determining the antimicrobial susceptibility of the bacteria in the sample. In some embodiments, the plurality of droplets each comprise a single bacterial cell from the sample. In some embodiments, the antimicrobial agent comprises an antimicrobial therapeutic agent. In some embodiments, the antimicrobial agent is selected from the group selected from: a carbapenem, a fluoroquinolone, a β-lactam, a aminoglycoside, trimethoprim/sulfmethoxazole, and the like. In some embodiments, the rRNA sequence comprises a 16S rRNA sequence. Typically, the methods disclosed herein include forming the droplets in a microfluidic device.

In some embodiments, the set of probes comprises a plurality of hybridization probes. In some embodiments, the set of probes comprises a plurality of hairpin probes. In some embodiments, a given probe comprises a first strand that comprises a peptide nucleic acid (PNA) molecule that comprises the nucleobase sequence that is at least partially complementary to the at least one region of the at least one bacterial rRNA sequence, and a second strand that comprises a nucleic acid molecule, wherein the second strand comprises a nucleobase sequence that is sufficiently complementary to a nucleobase sequence of the first strand such that the first and second strands hybridize with one another under conditions that are not sufficient for the probes to hybridize with the bacterial rRNA. In some embodiments, the first strand comprises a sequence length of about 15 to about 17 nucleobases and the second strand comprises a sequence length of about 10 to about 12 nucleobases. In some embodiments, the first strand comprises the reporter moiety and the second strand comprises the quencher moiety. In some embodiments, the first strand comprises the quencher moiety and the second strand comprises the reporter moiety. In some embodiments, the set of probes is selected from Table 1. In some embodiments, the at least one set of probes comprises at least first and second sets of probes that comprise reporter moieties that differ from one another. In some embodiments, the probes comprise one or more modified nucleobases. In some embodiments, the reporter moiety comprises at least one fluorophore.

In some embodiments, the reference detectable signal is obtained from droplets that comprise the bacterial cell and the set of probes and lack the antimicrobial agent. In some embodiments, a given droplet comprises a volume of about 10 picoliters (pL) or less. In some embodiments, a given droplet comprises between about 10³ and about 10⁵ copies of the rRNA. In some embodiments, the first time period comprises about 15 minutes or less. In some embodiments, the methods disclosed herein include incubating the set of droplets for the first time period at a temperature of about 37° C. In some embodiments, the second time period comprises about 20 minutes or less. In some embodiments, the methods disclosed herein include incubating the set of droplets for the second time period at a temperature of about 60° C. In some embodiments, the methods disclosed herein include lysing the bacterial cells in the droplets. In some embodiments, the methods disclosed herein include incubating the set of droplets for about two minutes at a temperature of about 95° C. to lyse the bacterial cells in the droplets.

In some embodiments, the bacterial cell comprises a Gram-negative bacterial cell. In some embodiments, the bacterial cell comprises an E. coli cell, a P. mirabilis cell, a K. pneumoniae cell, or a P. aeruginosa cell. In some embodiments, the bacterial cell is from the Enterobacterales order. In some embodiments, the bacterial cell is from the eubacterial kingdom.

In another aspect, the present disclosure provides a reaction mixture, comprising at least one set of probes selected from Table 1. In some embodiments, the reaction mixture disclosed herein further includes at least one antimicrobial agent. In some embodiments, the reaction mixture disclosed herein further includes at least one bacterial cell.

In another aspect, the present disclosure provides a device (e.g., a microfluidic device) that includes a body structure that defines at least one droplet formation cavity, at least one fluidic channel, and at least one detectable signal detection cavity. The droplet formation cavity and the detectable signal detection cavity fluidly communicate with one another via the fluidic channel. The droplet formation cavity fluidly communicates, or is capable of fluidly communicating, with at least one sample source, at least one reagent source, and at least one immiscible fluid source (e.g., an oil reservoir or the like). The reagent source comprises at least one set of probes in which the set of probes comprises a plurality of probes that each comprise at least one nucleobase sequence that is at least partially complementary to at least one region of at least one ribosomal RNA (rRNA) sequence of rRNA from at least one bacterial cell and in which the plurality of probes each comprise at least one reporter moiety and at least one quencher moiety that substantially quenches the reporter moiety at least when the plurality of probes are not hybridized to the rRNA. The droplet formation cavity is configured to form droplets that comprise at least some of the set of probes from the reagent source and at least an aliquot of a sample from the sample source. The fluidic channel comprises at least one droplet culture region, at least one bacterial cell lysis region, and at least one hybridization region, wherein the droplet culture region is configured to thermally communicate with a droplet culture thermal modulator, wherein the bacterial cell lysis region is configured to thermally communicate with a lysis thermal modulator, and wherein the hybridization region is configured to thermally communicate with a hybridization thermal modulator. The detectable signal detection cavity is configured to detectably communicate with one or more detectors that are capable of detecting detectable signals from reporter moieties of the probes when the probes hybridize with the rRNA from the at least one bacterial cell. The device is not configured to artificially amplify nucleic acids, if present, in or from samples or the aliquot of the sample from the sample source. In some embodiments, the set of probes is selected from Table 1. In some embodiments, a kit includes the device.

An embodiment of the present disclosure relates to the method of assembling a microfluidic device or cartridge as described herein fabricated from, for example, polymethylmethacrylate (PMMA), polypropylene (PP), polyethylene terephthalate (PET), polyethylene terephthalate glycol (PETG), high density polyethylene (HDPE), polytetrafluoroethylene (PTFE), polycarbonate (PC), and/or the like.

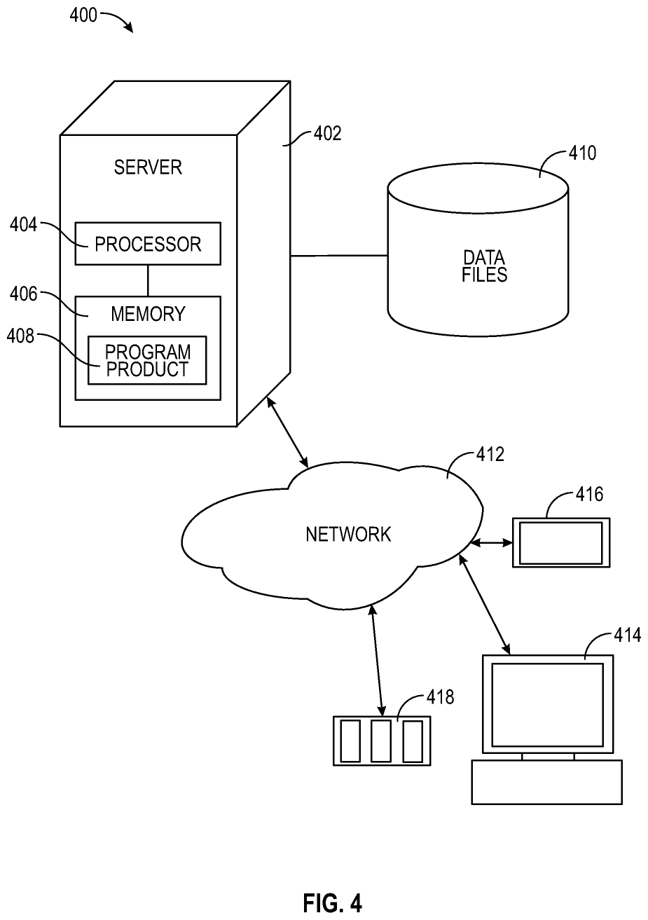

The present disclosure also provides various systems and computer program products or machine readable media. In some aspects, for example, the methods described herein are optionally performed or facilitated at least in part using systems, distributed computing hardware and applications (e.g., cloud computing services), electronic communication networks, communication interfaces, computer program products, machine readable media, electronic storage media, software (e.g., machine-executable code or logic instructions) and/or the like. To illustrate, FIG. 4 provides a schematic diagram of an exemplary system suitable for use with implementing at least aspects of the methods disclosed in this application. As shown, system 200 includes at least one controller or computer, e.g., server 402 (e.g., a search engine server), which includes processor 404 and memory, storage device, or memory component 406, and one or more other communication devices 414, 416, (e.g., client-side computer terminals, telephones, tablets, laptops, other mobile devices, etc. (e.g., for receiving captured images and/or videos for further analysis, etc.)) positioned remote from thermal modulating device 418 (e.g., including droplet culture, lysis, and hybridization thermal modulators for modulating temperatures in microfluidic devices when performing the methods disclosed herein), and in communication with the remote server 402, through electronic communication network 412, such as the Internet or other internetwork. Communication devices 414, 416 typically include an electronic display (e.g., an internet enabled computer or the like) in communication with, e.g., server 402 computer over network 412 in which the electronic display comprises a user interface (e.g., a graphical user interface (GUI), a web-based user interface, and/or the like) for displaying results upon implementing the methods described herein. In certain aspects, communication networks also encompass the physical transfer of data from one location to another, for example, using a hard drive, thumb drive, or other data storage mechanism. System 400 also includes program product 408 (e.g., related to an ocular pathology model) stored on a computer or machine readable medium, such as, for example, one or more of various types of memory, such as memory 406 of server 402, that is readable by the server 402, to facilitate, for example, a guided search application or other executable by one or more other communication devices, such as 214 (schematically shown as a desktop or personal computer). In some aspects, system 400 optionally also includes at least one database server, such as, for example, server 410 associated with an online website having data stored thereon (e.g., entries corresponding to more reference detectable signal strengths, indexed therapies, etc.) searchable either directly or through search engine server 402. System 400 optionally also includes one or more other servers positioned remotely from server 402, each of which are optionally associated with one or more database servers 410 located remotely or located local to each of the other servers. The other servers can beneficially provide service to geographically remote users and enhance geographically distributed operations.

As understood by those of ordinary skill in the art, memory 406 of the server 402 optionally includes volatile and/or nonvolatile memory including, for example, RAM, ROM, and magnetic or optical disks, among others. It is also understood by those of ordinary skill in the art that although illustrated as a single server, the illustrated configuration of server 402 is given only by way of example and that other types of servers or computers configured according to various other methodologies or architectures can also be used. Server 402 shown schematically in FIG. 4, represents a server or server cluster or server farm and is not limited to any individual physical server. The server site may be deployed as a server farm or server cluster managed by a server hosting provider. The number of servers and their architecture and configuration may be increased based on usage, demand and capacity requirements for the system 400. As also understood by those of ordinary skill in the art, other user communication devices 414, 416 in these aspects, for example, can be a laptop, desktop, tablet, personal digital assistant (PDA), cell phone, server, or other types of computers. As known and understood by those of ordinary skill in the art, network 412 can include an internet, intranet, a telecommunication network, an extranet, or world wide web of a plurality of computers/servers in communication with one or more other computers through a communication network, and/or portions of a local or other area network.

As further understood by those of ordinary skill in the art, exemplary program product or machine readable medium 408 is optionally in the form of microcode, programs, cloud computing format, routines, and/or symbolic languages that provide one or more sets of ordered operations that control the functioning of the hardware and direct its operation. Program product 408, according to an exemplary aspect, also need not reside in its entirety in volatile memory, but can be selectively loaded, as necessary, according to various methodologies as known and understood by those of ordinary skill in the art.

As further understood by those of ordinary skill in the art, the term “computer-readable medium” or “machine-readable medium” refers to any medium that participates in providing instructions to a processor for execution. To illustrate, the term “computer-readable medium” or “machine-readable medium” encompasses distribution media, cloud computing formats, intermediate storage media, execution memory of a computer, and any other medium or device capable of storing program product 408 implementing the functionality or processes of various aspects of the present disclosure, for example, for reading by a computer. A “computer-readable medium” or “machine-readable medium” may take many forms, including but not limited to, non-volatile media, volatile media, and transmission media. Non-volatile media includes, for example, optical or magnetic disks. Volatile media includes dynamic memory, such as the main memory of a given system. Transmission media includes coaxial cables, copper wire and fiber optics, including the wires that comprise a bus. Transmission media can also take the form of acoustic or light waves, such as those generated during radio wave and infrared data communications, among others. Exemplary forms of computer-readable media include a floppy disk, a flexible disk, hard disk, magnetic tape, a flash drive, or any other magnetic medium, a CD-ROM, any other optical medium, punch cards, paper tape, any other physical medium with patterns of holes, a RAM, a PROM, and EPROM, a FLASH-EPROM, any other memory chip or cartridge, a carrier wave, or any other medium from which a computer can read.

Program product 408 is optionally copied from the computer-readable medium to a hard disk or a similar intermediate storage medium. When program product 408, or portions thereof, are to be run, it is optionally loaded from their distribution medium, their intermediate storage medium, or the like into the execution memory of one or more computers, configuring the computer(s) to act in accordance with the functionality or method of various aspects. All such operations are well known to those of ordinary skill in the art of, for example, computer systems.

To further illustrate, in certain aspects, this application provides systems that include one or more processors, and one or more memory components in communication with the processor. The memory component typically includes one or more instructions that, when executed, cause the processor to modulate thermal modulator temperatures and effect detectable signal detection (e.g., via device 418) and/or receive information from other system components and/or from a system user.

In some aspects, program product 408 includes non-transitory computer-executable instructions which, when executed by electronic processor 404 perform at least: modulating temperatures of droplet culture thermal modulators, lysis thermal modulators, and hybridization thermal modulators, and effecting detection of detectable signals from the reporter moieties via operably connected detectors. Other exemplary executable instructions that are optionally performed are described further herein.

Additional details relating to computer systems and networks, databases, and computer program products are also provided in, for example, Peterson, Computer Networks: A Systems Approach, Morgan Kaufmann, 5th Ed. (2011), Kurose, Computer Networking: A Top-Down Approach, Pearson, 7^{th} Ed. (2016), Elmasri, Fundamentals of Database Systems, Addison Wesley, 6th Ed. (2010), Coronel, Database Systems: Design, Implementation, & Management, Cengage Learning, 11^{th} Ed. (2014), Tucker, Programming Languages, McGraw-Hill Science/Engineering/Math, 2nd Ed. (2006), and Rhoton, Cloud Computing Architected: Solution Design Handbook, Recursive Press (2011), which are each incorporated by reference in their entirety.

### EXAMPLE

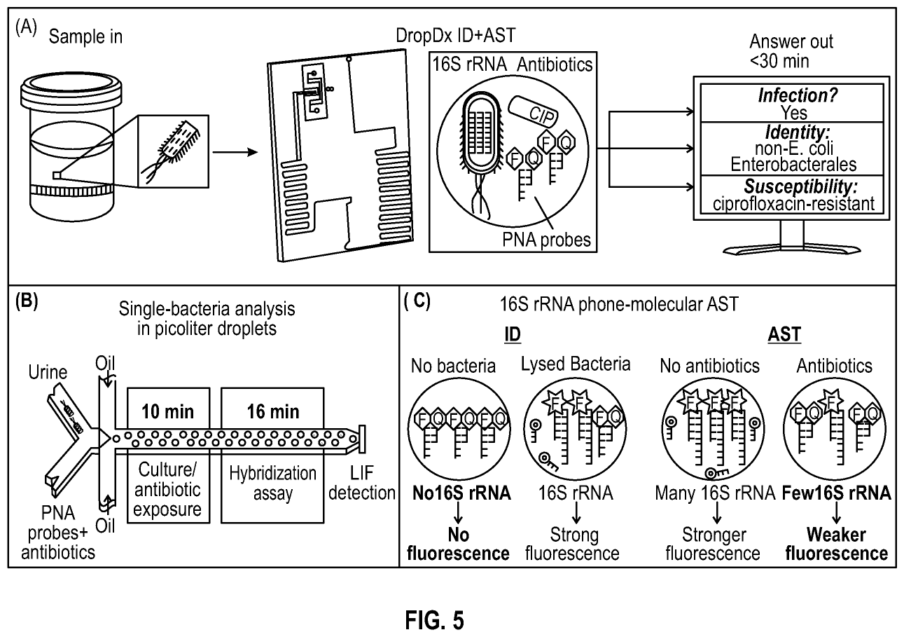

This example reports herein DropDx, the first UTI diagnostic platform that uses microfluidic droplet-based single-cell measurements of bacterial 16S rRNA to achieve both pathogen ID and AST from urine samples in as little as 30 minutes FIG. 5. In DropDx, each bacterial cell in a urine sample is co-encapsulated, within a picoliter droplet, along with an antibiotic and multiple fluorogenic peptide nucleic acid (PNA) probes (53-56) that target 16S rRNA sequences from different uropathogenic species or phylogenetic groups. Inside these droplets, bacteria are briefly exposed to the antibiotic, then thermally lysed, and further incubated to allow hybridization between complementary PNA probes and 16S rRNA. Upon detection, the fluorescence color of the droplet reveals the bacteria's ID and the fluorescence intensity of the droplet, which is proportional to the quantity of 16S rRNA within the droplet, reveals its susceptibility to the antibiotic. Such single-cell measurements of 16S rRNA enable DropDx to achieve unprecedented diagnostic speed, requiring as little as 16 minutes for hybridization and fluorescence-based ID and as little as 10 minutes antibiotic exposure for pheno-molecular AST against 3 common antibiotics with distinct mechanisms (i.e., gentamicin, ciprofloxacin, and ampicillin). The PNA probes employed in DropDx endows it with the capacity of classifying up to 9 unique uropathogenic species into appropriate phylogenetic categories that together account for ˜90% of UTI cases. As a demonstration of its clinical utility, 50 patient urine specimens were tested using DropDx and, in a fraction of the analysis time compared to clinical standard methods, acquired nearly identical ID and AST results (AUCs>0.95) and 95.3% categorical agreement.

### Materials and Methods

### Reference Bacteria, Antibiotics, and Urine Samples

Reference strains of E. coli (ATCC 25922), P. mirabilis (ATCC 12453), K. pneumoniae (ATCC BAA 1705), P. aeruginosa (ATCC 27853) as well as a multi-drug resistant strain of E. coli (ATCC BAA 2471) were all purchased from ATCC (Manassas, VA). Of note, the multi-drug resistant strain is reported to be resistant to a variety of drug classes including carbapenems, fluoroquinolones (e.g.: ciprofloxacin), β-lactams (e.g.: ampicillin), and aminoglycosides (e.g.: gentamicin). All bacterial strains were individually plated, and an isolated colony from each was grown until log phase in tryptic soy broth (TSB). The bacteria were then counted via plating in tryptic soy agar (TSA), and stocks were aliquoted and frozen with 20% glycerol (v/v) at −80° C. Prior to each experimental run, a fresh aliquot of bacteria was thawed and washed twice with Mueller-Hinton II cation-adjusted broth (MH) (Sigma-Aldrich, St. Louis, MO, USA). During experiments, bacteria were suspended in either MH or a mixture of 2×MH and urine.

### Design and Preparation of PNA Probes

PNA probes were designed to target specific regions in the 16S rRNA for organisms belonging to the Enterobacterales (EB) order, the E. coli and P. mirabilis species (EC and PM), and the eubacterial kingdom (UNI), based on previous work (52, 72) (Table 1). The EB, EC, and PM probes were labeled with a FAM fluorophore, whereas the universal probe was labeled with an Alexa546 fluorophore. For each PNA probe, a short complementary DNA quencher was designed labeled with an Iowa Black® dark quencher. All probes were synthesized by PNA Bio (Newbury Park, CA), and all DNA quencher strands were synthesized by Integrated DNA Technologies (San Diego, CA). Probes and quenchers were stored in a custom hybridization buffer consisting of 50 mM NaCl, 25 mM Tris-HCl pH 8.0, and 0.5% polyvinylpyrrolidone (PVP) at −20° C. PVP is included to reduce the likelihood of probes being adsorbed to or retained by the walls of the PDMS devices. Prior to each experiment, individual aliquots of PNA probes were first thawed at 60° C. for 10 min to ensure full dissolution of the PNA stock into the hybridization buffer. For both bulk-based and droplet experiments, 200 nM PNA probes were mixed with 600 nM of the corresponding quencher in the custom hybridization buffer, and this solution was either mixed in bulk or co-encapsulated into droplets at a 1:1 ratio along with bacteria-containing urine and/or MH suspensions. An appropriate dosage of antibiotics was included in this mixture if required for the experiment.

TABLE 1
Summary of designed PNA Probes. Four unique PNA probes were
designed and tested, specific to the species E. coli and P. mirabilis,
the Enterobacterales order, and the bacterial kingdom (eubacteria).
Custom DNA quenchers were designed tagged with an Iowa-Black quencher
that spanned 10-11 complimentary bases to the PNA probes.

| Probe | Target | Sequence | Tₘ (° C.) |
|---|---|---|---|
| EC | E. coli | FAM-O-TCAATGAGCAAAGGT-KK | 76.3 |
| | | IABk-AGTTACTCGT | |
| PM | P. mirabilis | FAM-O-TCCTCTATCTCTAAAGG | 67.2 |
| | | IABk-AGGAGATAGAG | |
| EB | Enterobacterales | FAM-O-TATGAGGTCCGCTTG | 73.4 |
| | | IABk-ATACTCCAGG | |
| UNI | Eubacteria | GCTGCCTCCCGTAGGA-K-Alexa546 | 80.5 |
| | | GGAGGGCATCCT-IABk | |

### Design and Fabrication of Microfluidic Devices

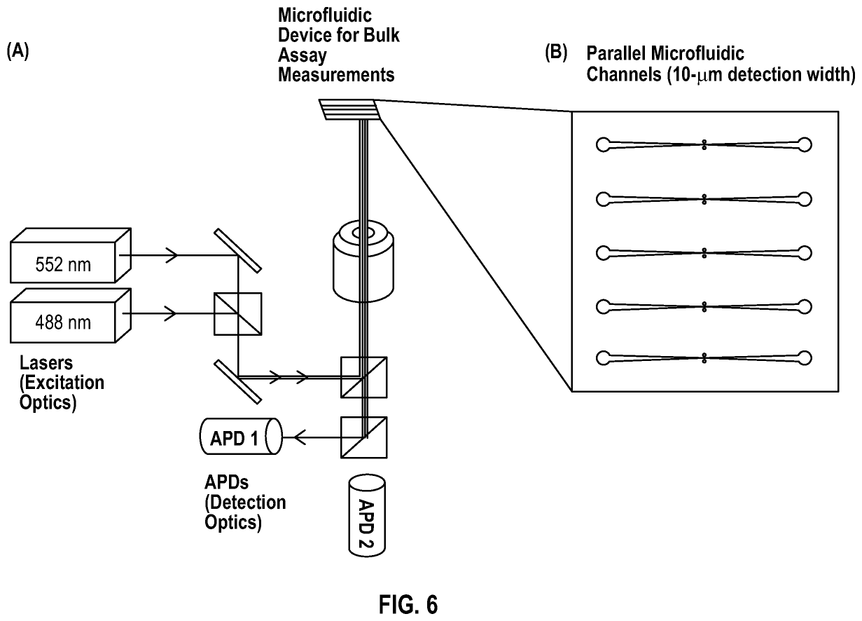

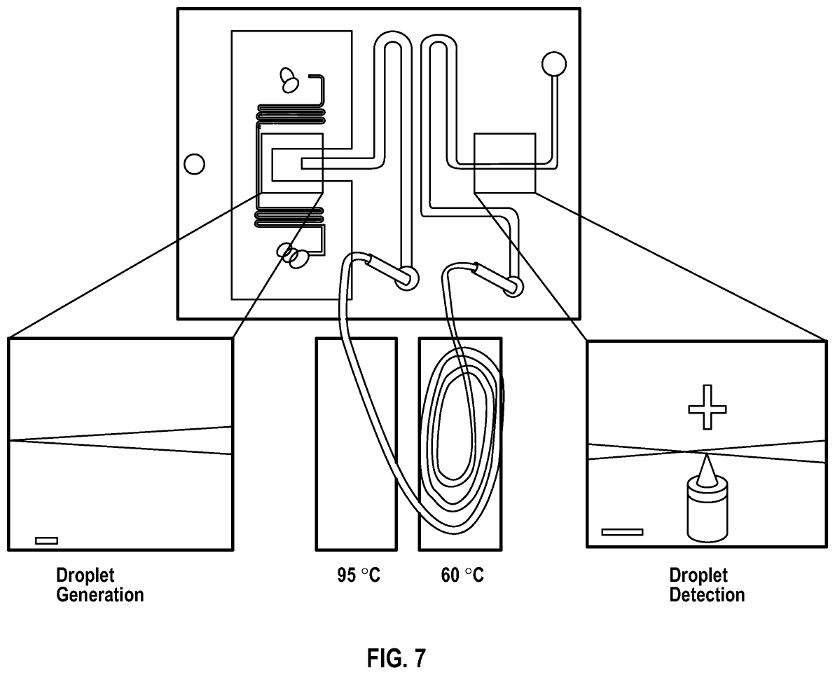

Three distinct microfluidic devices were used in this work—2 for assay characterization and 1 for clinical testing and validation of the DropDx workflow. The first microfluidic device was used for bulk-based characterization of the PNA probe assay, and consists of an array of single channels, each with a 10-μm-wide×20-μm-tall constriction for laser induced fluorescence (LIF) detection of benchtop assay reactants (FIG. 6). For assay characterization in droplets, a droplet device was designed that consisted solely of a droplet generation module and a droplet detection module. The two modules were connected by Tygon tubing of variable length, which enabled testing of varying hybridization durations (FIG. 7). This device consisted of a 10-μm-wide×20-μm-tall flow-focusing nozzle for droplet generation and an equidimensional constriction for droplet detection. Casting molds for both characterization devices were constructed by spinning a 20 μm layer of SU8-3050 onto a 4 inch silicon wafer and patterning using standard photolithography. In order to test larger and smaller droplet volumes, the height of the droplet generation module was increased or lowered appropriately by spinning a thicker or thinner layer of SU8-3050 photoresist.

Following assay characterization, an integrated DropDx device was designed that consists of a droplet generator capable of producing 4 pL droplets, a culture/drug exposure channel that houses droplets for 10 minutes, a lysis channel that houses droplets for 2 minutes, a hybridization channel that houses droplets for 14 minutes, and a droplet detection constriction. The DropDx device consists of a 10-μm-wide and 20-μm-tall flow-focusing nozzle for droplet generation, followed by a 1000-μm-wide×60-μm-tall×169-mm-long serpentine incubation channel for bacterial culture/drug exposure, a 1000-μm-wide×60-μm-tall×33-mm-long channel for bacterial lysis, and a 1000-μm-wide×60-μm-tall×248-mm-long channel for probe hybridization. Finally, the channels narrow into a 10-μm-wide and 20-μm-tall droplet detection window before widening back into an outlet. A casting mold was fabricated by first spinning a 20 μm layer of SU8-3050 photoresist (MicroChem) onto a 4 inch silicon wafer and patterning only the droplet generation and droplet detection regions using standard photolithography. Next, a 60 μm layer of SU8-3050 was spun onto the same mold in order to create the 3 incubation regions. The photomask of the incubation channels was aligned to the mold using microscopy in order to ensure continuity from droplet generation to droplet detection. Patterning of the second layer was completed using standard photolithography.

All microfluidic devices were created by pouring 30 g of 10:1 ratio of polydimethylsiloxane (PDMS) Sylgard 184 (Dow Corning) base to curing agent onto the silicon/SU8 mold. After curing the PDMS replica, holes were punched onto the device inlet ports and the device outlet port. The PDMS replica was then permanently bonded to cover glass (130 μm thickness, Ted Pella) through oxygen plasma treatment in order to seal the channels. Prior to device operation, all microfluidic chips were treated with Aquapel and baked at 80° C. for at least 20 minutes to render microfluidic channel surfaces hydrophobic.

### Operation of Microfluidic Platform

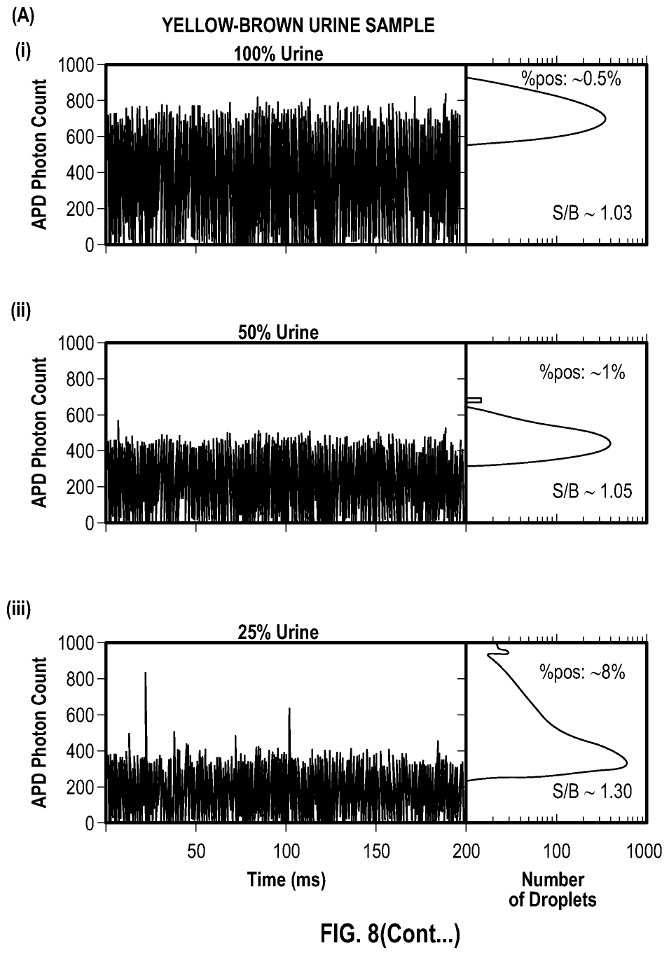

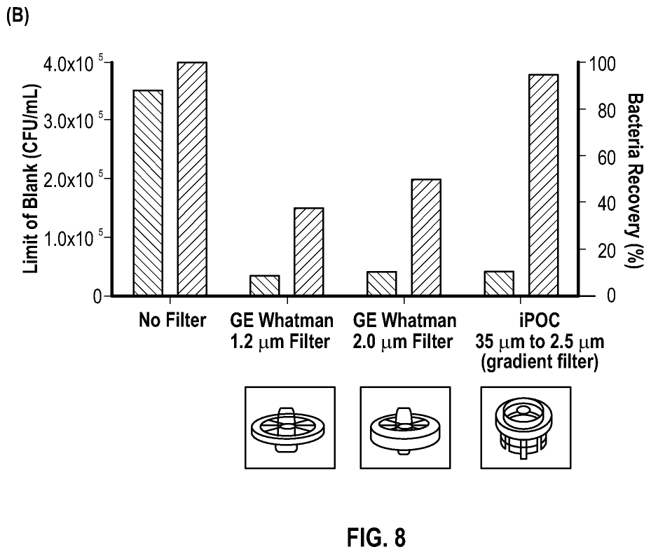

One-step pretreated urine samples (FIG. 8) and probe/quencher mix (without or with antibiotics) were first introduced into separate sections of Tygon tubing. Both sections of Tygon tubing were individually connected to Hamilton 1000 glass syringes (Sigma-Aldrich) containing FC-40 oil (Sigma-Aldrich). FC-40 oil in the syringe was used to push the aqueous samples from Tygon tubing into the device using a syringe pump (Harvard Apparatus). BioRad QX200 Droplet Generation Oil (BioRad Laboratories) was introduced into the oil inlet of the device by a separate syringe pump. A flow rate of 15 μL h⁻¹ was used for both aqueous phases, while 60 μL h⁻¹ was used for the oil phase. In order to confirm stable and uniform droplet generation, the device was imaged using a 4× objective lens and a CCD camera during droplet generation and after droplet incubation. Generated droplets flowed through Tygon tubing connecting the droplet generation and droplet detection regions of the microfluidic device. Importantly, separate lengths of the Tygon tube were clamped onto a 95° C. Peltier heater and a 60° C. Peltier heater, such that all droplets can flow through the lysis and hybridization regions for the appropriate duration required for the experiment. Droplets traveling at 60 μL h⁻¹ traverse 1 cm through the Tygon tube every 2 minutes. Thus, to ensure 2 minutes lysis and 30 minutes hybridization, 1 cm of the Tygon tubing was clamped to the 95° C. heater and 15 cm of the tubing to the 60° C. heater.

### Droplet Fluorescence Detection

Continuous-flow droplet measurements in 2 colors was conducted using an LIF detector that consists of an optical stage which was interfaced with 488 nm and 552 nm laser excitation sources (OBIS, Coherent, Inc.) and 2 silicon avalanche photodiode detectors (APD) (SPCM-AQRH13, ThorLabs). The 488 nm laser was operated at 4 mW power and the 552 nm laser was operated at 10 mW power to ensure sufficient illumination and maximize signal over background from each fluorophore. Both lasers were focused into the detection zone of the device using a 40× objective (Thorlabs RMS40X-PF, NA 0.75, focal depth approximately 0.6 μm). As droplets flowed through the custom laser-induced fluorescence detection zone, fluorescence data was continuously acquired and recorded using the APDs with 0.1 ms sampling time. A custom LabVIEW program was used to display and save APD measurements in real time.

A custom MATLAB program was developed for analyzing the raw fluorescence intensity data acquired from the APD. From the fluorescence intensity time trace of each experimental run, the program looks for individual droplets by quantifying peak widths and peak heights. Once droplet position and fluorescence intensity were identified for all droplets in a sample, the intensities were plotted as a histogram with ˜150 bins. Resulting histograms typically follow a bimodal distribution, and in order to classify the respective subpopulations, the first droplet histogram peak (“empty droplets”) were fitted with a Gaussian curve. Based on the fitted curve, the mean intensity of the empty droplet population as well as the standard deviation were calculated. All droplets whose intensities were 5 standard deviations above the empty droplet mean intensity were counted as bacteria-containing “positive droplets.” Signal to background ratios were calculated by dividing the mean intensity of positive droplets by the mean intensity of empty droplets.

### Statistical Analyses and Curve Fitting

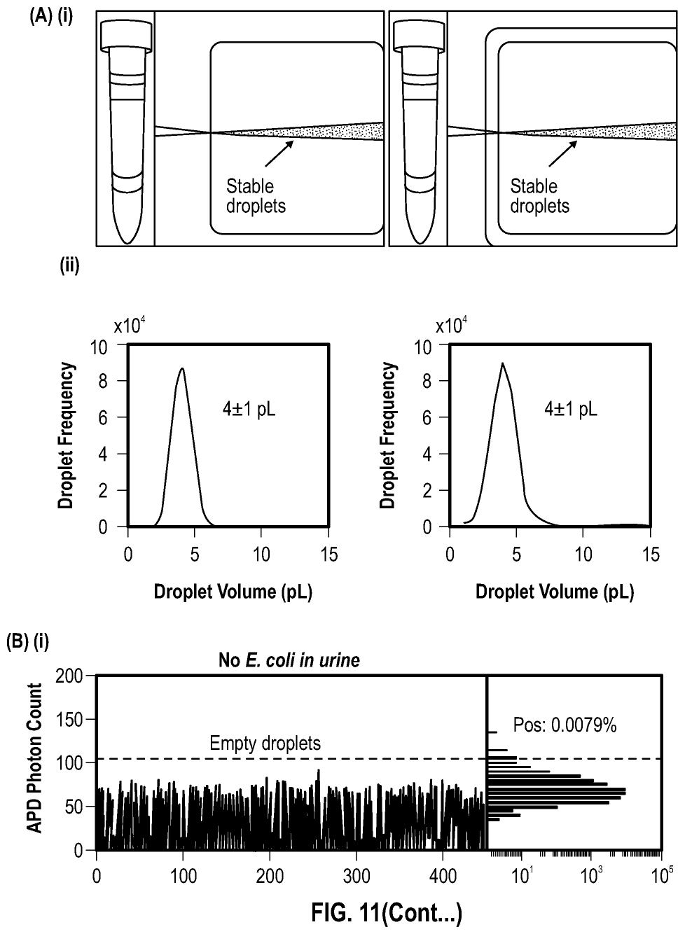

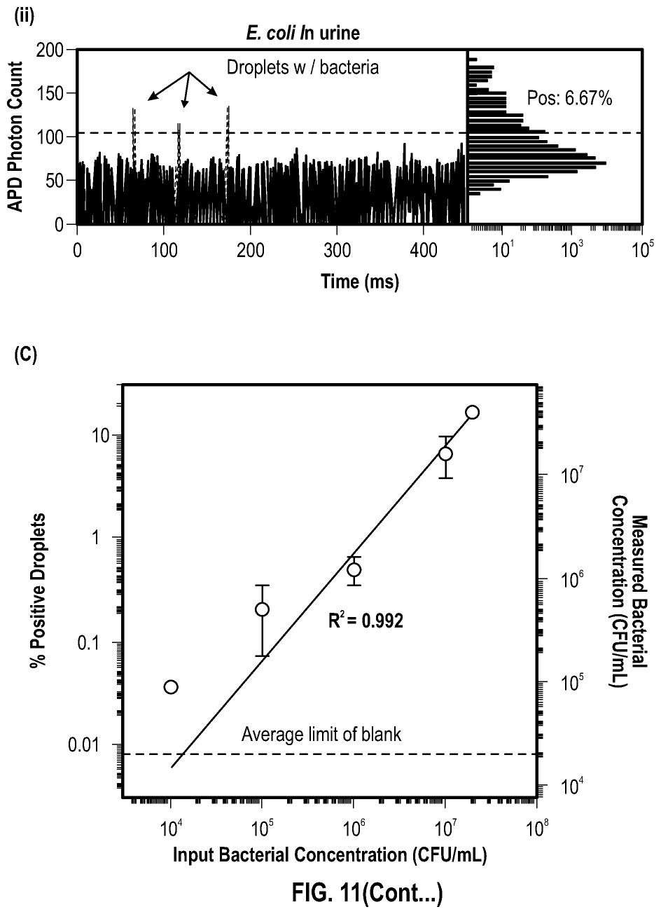

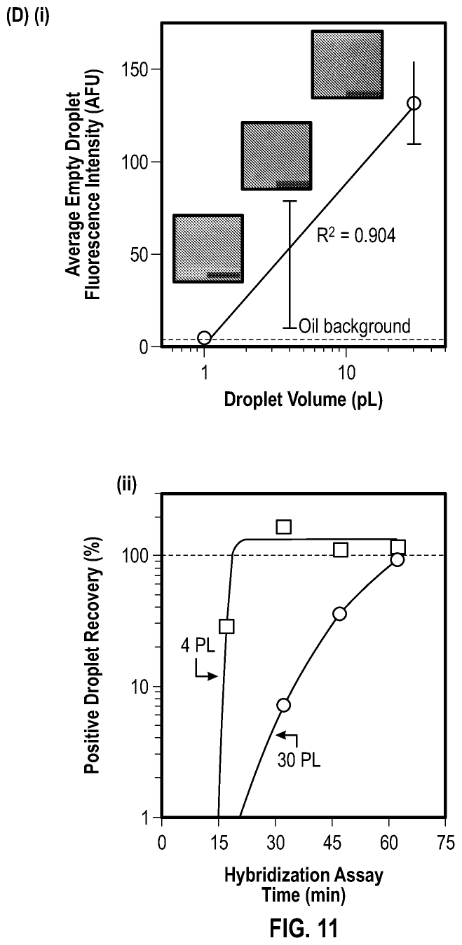

All reported p-values in this work were calculated by performing unpaired one-tailed t-tests in GraphPad Prism (GraphPad Software Inc., San Diego, CA). GraphPad was also used to fit curves for droplet data in FIGS. 11C and 11D and for extracting relevant fit parameters.

### Validation of DropDx Platform With Clinical Urine Specimens

Fifty UTI specimens were obtained and tested under an approved institutional review board (IRB) study at the Johns Hopkins University School of Medicine (JHU-SOM) (IRB00189525). All tested specimens, obtained from the Johns Hopkins Hospital (JHH) Clinical Microbiology Laboratory, were also subject to the standard clinical specimen management workflow. In the standard workflow, 1 to 10 μL of each specimen is plated in order to achieve clinical isolation of the uropathogen, and the remaining urine specimen is refrigerated. Typically, within 8 h of plating, the relative uropathogenic load can be estimated by direct observation of the plate. For DropDx, refrigerated urine specimens whose plates displayed a relatively high pathogen load (estimated >30,000 CFU/mL) of a single dominant uropathogen were selected. At JHH, each clinically isolated bacteria specimen was identified using MALDI-TOF mass spectrometry Bruker Daltonics, Inc., Billerica, MA) and subject to AST using BD Phoenix (BD Diagnostics, Sparks, MD) enabled broth microdilution, processes that required up to 48 h of turnaround. The corresponding refrigerated specimens were tested with DropDx right after selection and required less than 30 min of device operation.

In order to ensure sample freshness, all refrigerated urine specimens were tested in the DropDx platform no more than 1-2 days after collection from the patients. Early in the clinical data validation process, it was chosen to work with urine specimens that contained bacteria-preserving additives. Urine specimens at HIE were collected from patients in either a sterile cup or a BD Vacutainer urine collection transport tube (BD Diagnostics, Sparks, MD, #364951). Typically, cup samples do not contain any additives, whereas the much more common Vacutainer tubes include a mixture of boric acid, sodium formate, and sodium borate. Upon confirming that comparable results can be observed from samples without and with the preservatives, predominantly additive-containing specimens were tested due to their significantly higher abundance and accessibility within the clinical laboratory.

### Threshold Criteria and Power Analysis for DropDx Clinical Comparison Study

ROC curve and statistical power analyses were implemented to ensure unbiased thresholding and sufficient statistical confidence for data generated. The strategy consisted of using the initial 15 specimens for an AST pilot study, the next 16 specimens concurrently for an ID pilot study and AST validation study, and the final 19 specimens for both ID validation study and AST validation study. Culture-negative and culture-positive specimens were differentiated from each other by the total percentage of positive droplets (across FAM and Alexa546 channels) from the no-antibiotic control (the first DropDx device). EC-containing specimens were differentiated from non-EC specimens by the ratio of UNI-positive droplets to EC-positive droplets from the first device. Non-EC EB specimens were differentiated from EB specimens by the percentage of EB-positive droplets (multiplied by the dilution factor) from the second DropDx device. Finally susceptible specimens were differentiated from resistant specimens by the ratio of UNI-positive droplets from the second and first DropDx devices. For each category call in the platform (i.e., culture-negative vs culture-positive, EC vs non-EC, EB vs non-EB, and susceptible vs resistant), ROC curves were plotted using data from the appropriate pilot study using GraphPad Prism. Next, an optimal threshold was established by plotting the Youden's Index from each ROC curve, derived by calculating the sensitivity+specificity−100%, at various ROC thresholds, and then extracting the global maximum value. This threshold was kept constant for all subsequent data collected in the validation studies. Comprehensive raw data from each tested specimen is presented in Table 2.

TABLE 2
Clinical performance (PPVs and NPVs) of DropDx ID and AST. The true positive,
false positive, true negative, false negative, PPVs, and NPVs are tabulated for
DropDx readouts for (A) culture negative/positive, (B) EC negative/positive,
(C) EB negative/positive, and (D) ciprofloxacin resistant/susceptible metrics.

| | Standard Clinical Readout | | | | |
|---|---|---|---|---|---|
| | Culture | Culture | | Standard Clinical Readout | |
| (A) | Positive | Negative | (B) | EC Positive | EC Negative |
| DropDx | 27 | 0 | DropDx | 13 | 0 |
| Culture Positive | | | EC Positive | | |
| DropDx | 1 | 7 | DropDx | 0 | 15 |
| Culture Negative | | | EC Negative | | |
| | PPV: | NPV: | | PPV: | NPV: |
| | 27/28 = 96.4% | 7/7 = 100% | | 13/13 = 100% | 15/15 = 100% |

| | | | | Standard Clinical Readout | |
|---|---|---|---|---|---|
| | Standard Clinical Readout | | | Cipro | Cipro |
| (C) | EB Positive | EB Negative | (D) | Resistant | Susceptible |
| DropDx | 20 | 1 | DropDx | 14 | 2 |
| EB Positive | | | Cipro Resistant | | |
| DropDx | 0 | 7 | DropDx | 0 | 27 |
| EB Negative | | | Cipro Susceptible | | |
| | PPV: | NPV: | | PPV: | NPV: |
| | 20/20 = 100% | 7/8 = 87.5% | | 14/14 = 100% | 27/29 = 93.1% |

Power analyses of pilot datasets were conducted by calculating statistical power achieved for varying sample sizes for each category call in MATLAB. The samplesizepwr function was used, and input the average and standard deviation values from each pilot dataset along with a desired confidence of 95% (α=0.95). Power vs sample size curves were then plotted in MATLAB, followed by a linear interpolation of the number of samples required to reach a power of 90% (1-β).

### Results

DropDx ID and Pheno-Molecular AST Enabled by Single-Cell Detection of 16S rRNA in Picoliter Droplets

In realizing DropDx, microfluidic droplet-based single-cell detection and 16S rRNA-based pheno-molecular AST were combined into a one-step assay within an integrated platform. To do so, microfluidic flow-focusing were employed to confine single bacterial cells in urine into picoliter droplets along with fluorogenic PNA probes. Such confinement can reduce fluorescence background from the urine sample matrix and enhance binding of probes and targets due to the increased concentration of sub-cellular 16S rRNA from single cells, and can therefore reduce the turnaround time for bacterial detection. The generated droplets traverse heated regions designed to facilitate bacterial culture/antibiotic exposure for as short as 10 min and thermal lysis and PNA-to-16S rRNA hybridization (together, the “hybridization assay”) for as short as 16 minutes before being individually measured for fluorescence on-chip by a custom 2-color laser-induced fluorescence (LIF) detector (FIG. 5B). Whereas fluorophore-labeled PNA probes in empty droplets or droplets without target bacteria remain bound to a short quencher-tagged complementary DNA sequence, in the presence of target bacteria, PNA probes competitively hybridize to complementary 16S rRNA targets, releasing them from their quenchers and producing a strong fluorescence signal. The fluorescence emitted from each droplet is measured using two fluorescence channels, and the resulting color signature is used to identify and classify the uropathogen (FIG. 5C, “ID”). As antibiotic-free cells produce a greater quantity of 16S rRNA after a short culture than susceptible antibiotic-dosed cells (and comparable quantity of 16S rRNA as resistant antibiotic-dosed cells), the difference in droplet fluorescence intensities between these two conditions can be used to assess the bacterial responses to antibiotic treatment and determine the susceptibility (FIG. 5C, “AST”).

PNA Probe-Based Hybridization Assay for Detection of Bacterial 16S rRNA in Urine

For designing the fluorogenic probes in the hybridization assay, we first targeted 16S rRNA from predominant UTI-causing pathogens, then considered its chemical structure and molecular configuration, and finally its compatibility with the LIF detector. First, we designed our probes to target E. coli, P. mirabilis, and the Enterobacterales order, which encompasses the two aforementioned species and other important uropathogenic genera such as Klebsiella, Citrobacter, and Serratia. Our E. coli (EC) probe, P. mirabilis (PM) probe, and Enterobacterales (EB) probe together account for ˜75%, ˜3%, and ˜87% of UTI cases (1, 57), respectively.

Additionally, we designed a pan-bacteria, universal (UNI) probe for confirming or ruling out bacterial infection, which accounts for nearly 100% of UTI cases (Table 1). Next, we employed PNA in the probe because the neutrally-charged polyamine backbone of PNA (53-56) is thermally stable and resistant to various pH and salt concentrations, and thus well suited for diagnostic assays in urine samples. We used a hybrid probe configuration composed of a fluorophore-labeled PNA strand (15-17 nt) and a short complementary quencher-tagged DNA strand (10-12 nt). Hybridization of the PNA strand to bacterial 16S rRNA target simultaneously displaces the DNA quencher strand, which liberates probe fluorescence and facilitates robust bacteria detection (52). Finally, because the LIF detector is equipped to detect FAM and Alexa546, we tagged our PNA strand with either FAM or Alexa546 fluorophores and the DNA strand with corresponding quenchers.

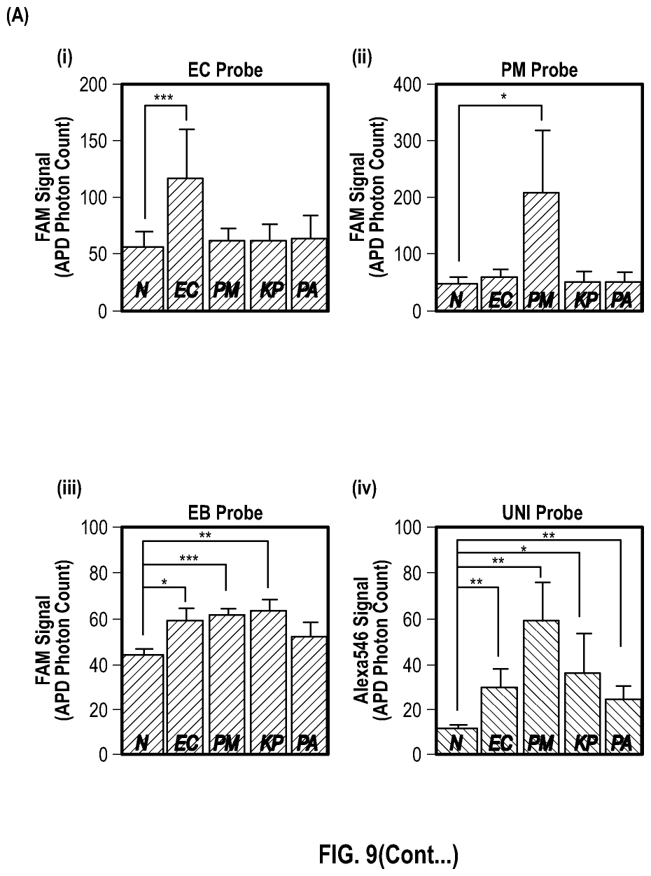

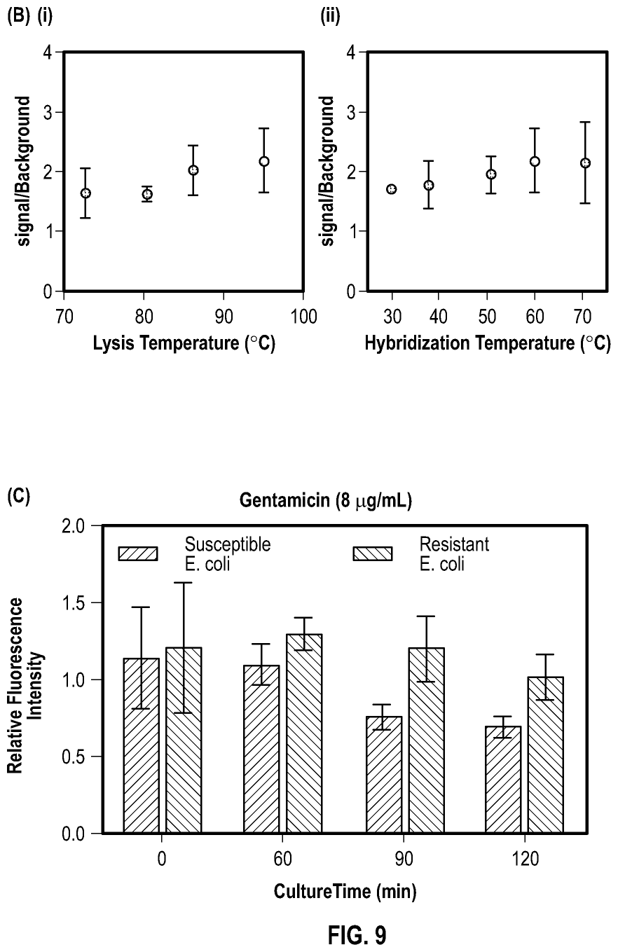

We first verified that the probes can specifically detect target bacteria spiked into urine. In these bulk-based (20 μL volume) experiments, we ensured that the assay conditions, including the bacterial concentration, the detection channel, and the detector, were comparable to droplet-based detection. We spiked ˜10⁹ CFU/mL (equivalent to 1 cell in a ˜1 pL droplet) of standard reference strains of E. coli ATCC 25922, P. mirabilis ATCC 12453, K. pneumoniae ATCC BAA 1705, and P. aeruginosa ATCC 27853 into a mixture of culture-negative (i.e., blank) urine samples and hybridization buffer with each PNA probe. Each sample was subjected to 2 min of thermal lysis at 95° C. and 30 min of hybridization at 60° C., and then detected at room temperature within a 10 μm-wide microfluidic detection channel by our 2-color LIF detector (FIG. 6). As expected, the FAM-labeled EC probe (FIG. 9Ai) and the FAM-labeled PM probe (FIG. 9Aii) yield significantly higher signal in the presence of E. coli and P. mirabilis, respectively. The FAM-labeled EB probe yields significantly higher signals for E. coli, P. mirabilis, and K. pneumoniae—all within the Enterobacterales order (FIG. 9Aiii), while the Alexa546-labeled UNI bacterial probe successfully detects all of the tested uropathogens (FIG. 9Aiv). Of note, using the EC probe and the reference E. coli strain, we also found that the assay works across a wide range of lysis temperatures (FIG. 9Bi) and hybridization temperatures (FIG. 9Bii). These results demonstrate that the probes are target-specific, functional in urine, consistent at various temperatures, and can be readily implemented within droplets.

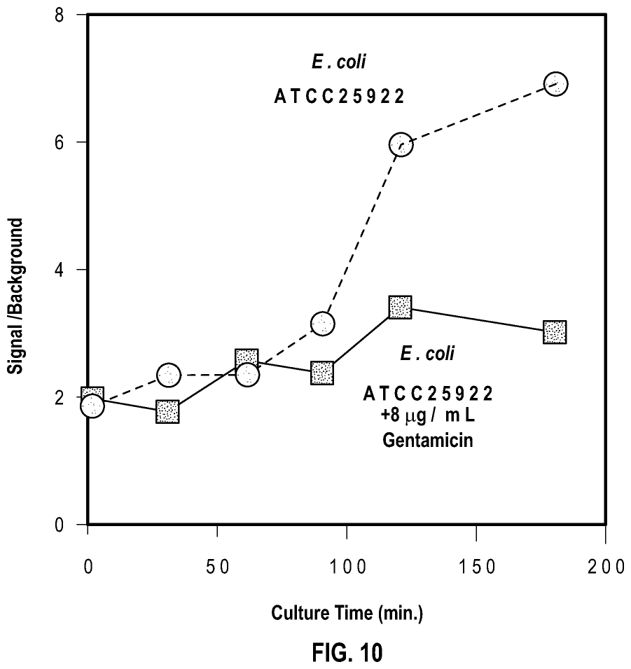

We note that we can achieve preliminary pheno-molecular AST by directly detecting PNA-to-16S rRNA hybridization in the bulk format via the LIF detector, though only after relatively lengthy antibiotic exposure. To demonstrate, we incubated EC PNA probes with either multi-drug resistant E. coli ATCC BAA 2471 or the reference E. coli strain, each strain without and with gentamicin (at a bactericidal concentration of 8 μg/mL) in 20 μL sample volume for 0, 60, 90, or 120 min at 37° C. before subjecting these samples to 2-min 95° C. lysis, 30-min 60° C. hybridization, and LIF detection within 10-μm-wide detection channels. We then determined the relative fluorescence intensity—the ratio between the fluorescence intensities of the gentamicin-dosed cells and the gentamicin-free cells—for both strains for each culture duration. For the multi-drug resistant E. coli, the relative fluorescence intensity remained close to 1, indicating that gentamicin-dosed cells and gentamicin-free cells produced similar amounts of 16S rRNA, which corroborates its resistance to gentamicin (FIG. 9C, red). In contrast, for the reference E. coli, which is susceptible to gentamicin, gentamicin-dosed cells were inhibited from growth and consequently produced a markedly lower quantity of 16S rRNA than gentamicin-free cells over time (FIG. 10), allowing us to detect significantly lower relative fluorescence intensities compared to the multi-drug resistant E. coli after 90 min (FIG. 9C, green). These results confirm the feasibility of pheno-molecular AST using the fluorogenic probe hybridization assay and pave the way for implementing the assay in droplets.

In-Droplet 16S rRNA-Based Detection of Single Bacterial Cells From Urine Samples

For achieving and characterizing 16S rRNA-based single-bacteria detection within droplets generated from urine samples, we established a simple urine pretreatment protocol, and we set up a modular microfluidic device (FIG. 7) to flexibly characterize assay time requirements. Impurities in urine samples, mostly μm-to-mm salt crystals, casts, and cellular debris, can clog devices and hinder droplet generation. These impurities can also autofluoresce and produce spurious fluorescence signals in droplets, obfuscating droplet analysis. Moreover, the quantity of these impurities in urine samples can vary considerably by patients and sample collection processes (58). We therefore implemented a simple protocol in which all urines samples were filtered through a gradient-based syringe filter (iPOCdx, 35 μm to 2.5 μm) and diluted four-fold in MH broth prior to droplet generation and analysis (FIG. 8). These filtered and diluted urine samples were then co-injected with PNA probes into the modular devices. Droplets containing single bacterial cells and PNA probes first formed in the droplet generation unit of the device, then flowed out of the device into a Tygon tubing that was placed on a 95° C. Peltier heater for bacterial lysis and a 60° C. Peltier heater for PNA-to-16S rRNA hybridization, and finally re-entered the device and through the 10 μm-wide detection channel, where their fluorescence signals were measured sequentially by the LIF detector (FIG. 7). This modular device is particularly useful for optimizing the lysis and hybridization times, as we would simply adjust the length of Tygon tubing instead of designing and fabricating new devices.

We next demonstrated successful in-droplet 16S rRNA-based detection of single bacterial cells from urine samples. As an important prerequisite, we first showed that we could stably generate monodisperse, 4±1 pL droplets from various urine samples (ranging from visibly light yellow/clear to turbid orange) without clogging (FIG. 11A). For demonstrating single-cell detection, we co-encapsulated EC probes and blank urine samples spiked without and with E. coli cells (2×10⁷ CFU/mL) into droplets, heated these droplets at 95° C. for 2 min and 60° C. for 30 min, and detected these droplets using the LIF detector. The urine sample without E. coli yielded low-intensity fluorescence peaks in the fluorescence peak trace that correspond to empty droplets (FIG. 11Bi). In contrast, the urine sample with E. coli yielded additional high-intensity fluorescence peaks in the fluorescence peak trace that indicate the presence of E. coli in these droplets (heretofore, “positive” droplets) (FIG. 11Bii, green). Importantly, droplet fluorescence intensity histograms revealed only 0.0079% of (false) positive droplets (likely from autofluorescent impurities) in the urine sample without E. coli (FIG. 11Bi) but ˜6.67% of positive droplets in the urine sample with E. coli (FIG. 11Bii), which corresponds to ˜3.34×10⁷ CFU/mL. The reasonable agreement between the spike-in concentration and the measured concentration suggests that E. coli was indeed individually detected.

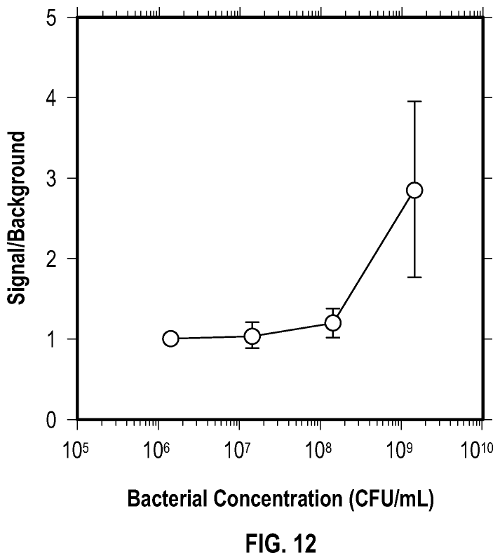

We subsequently ensured that we can detect clinically relevant bacterial loads for UTI ranging from 10⁴ CFU/mL to 10⁷ CFU/mL using the in-droplet 16S rRNA-based detection. Toward achieving this, the urine pre-treatment protocol helped reduce autofluorescence in urine samples and significantly lower the limit of blank (i.e., the number of false-positive droplets from blank urine samples), while allowing ˜94% recovery of bacteria from the samples (FIG. 8). Aided by the low limit of blank and high recovery of bacteria, we successfully detected 2×10⁷, 10⁷, 10⁶, 10⁵, and 10⁴ CFU/mL of the reference E. coli strain spiked in blank urine samples with high linearity (FIG. 11C, R²=0.992). Of note, the droplet-based assay was ˜5 orders of magnitude more sensitive than its bulk-based counterpart (FIG. 12), which not only highlights an important advantage of in-droplet assays, but also suggests that a similar improvement in pheno-molecular AST can be expected.

For in-droplet assays, reduction in droplet volume would simultaneously increase the target concentration and decrease the background noise in each droplet, thus enhancing the signal-to-background ratio and potentially shortening the assay time (59-61). As we aim to develop a rapid diagnostic tool, we sought to shorten the assay time by testing our assay within 30 pL, 4 pL, and 1 pL droplets (generated via distinct modular devices with increasingly shallow microfluidic channels). We first verified robust generation of droplets at these 3 volumes from blank urine samples (FIG. 11Di; n=3 for 30 pL, 4 pL, n=2 for 1 pL). As these no-bacteria, empty droplets shrank from 30 pL to 1 pL, the average background fluorescence intensity decreased and eventually became undetectable from that of the oil phase. Because the detection method relies on calculating the percentage of positive droplets from all droplets, which necessitates accurate detection of empty droplets, we excluded 1 pL droplets from further characterization. Next, we co-spiked E. coli and the EC PNA probe into blank urine samples, generated 30 pL and 4 pL droplets, and gradually shortened the PNA-to-16S rRNA hybridization time from 60 min to 15 min. For each set of droplets, we compared the expected rate of positive droplets, based on the spike-in concentration of bacteria, with the measured rate of positive droplets (“positive droplet recovery”) and found that the assay was indeed faster within 4 pL droplets, detecting the expected amount of single bacterium-containing droplets (FIG. 11Dii, 100% “positive droplet recovery”) after 30 min hybridization, compared to 60 min within 30 pL droplets. In 4 pL droplets, we could still detect single E. coli cells after only 15 min of hybridization (FIG. 11Dii). Our focus on achieving a rapid assay, coupled with our approach of comparing the relative change between samples with and without antibiotics, prompted us to employ 15 min as the hybridization time in our in-droplet assays hereafter.

Rapid Assessment of Bacterial Susceptibility to Antibiotics Via Quantitative Detection of 16S rRNA From Single Cells

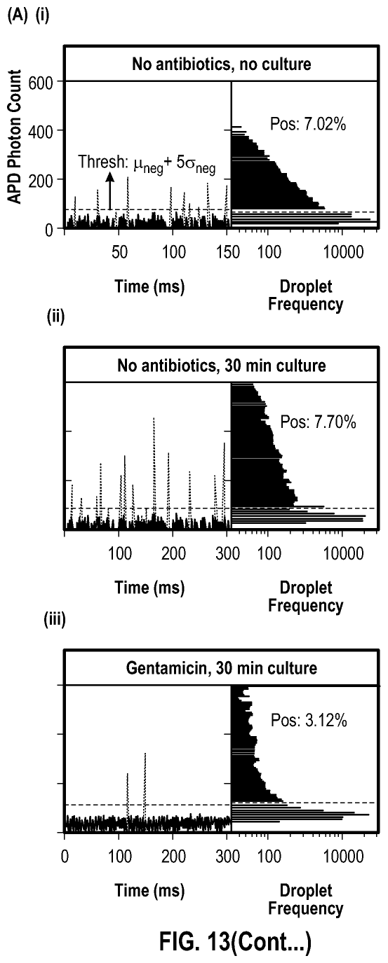

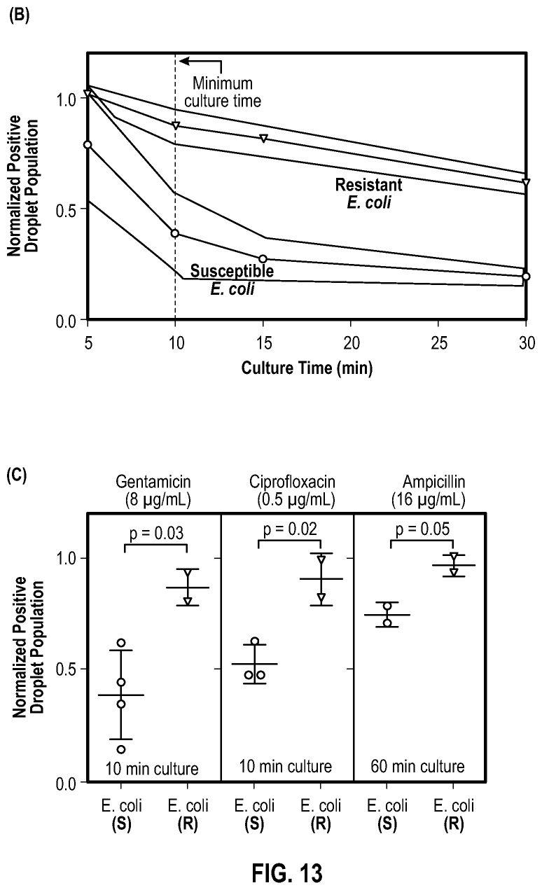

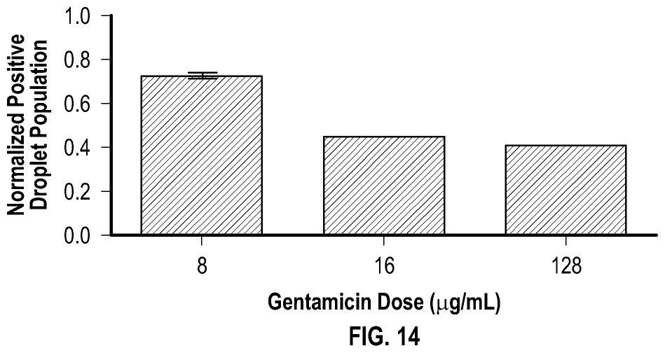

As initial steps toward achieving in-droplet pheno-molecular AST via measurements of 16S rRNA of single bacterial cells, we first demonstrated the detection of bacterial growth via increased 16S rRNA production within droplets and subsequently demonstrated inhibited bacterial growth and reduced production of 16S rRNA within droplets due to antibiotic exposure. We first co-encapsulated the EC probe and E. coli cells, which were spiked into Mueller-Hinton broth (MH), into picoliter droplets. One set of generated droplets was subject to only the hybridization assay (i.e., 95° C. for 2 min for bacterial lysis, and 60° C. for 15 min for hybridization), while the other set of droplets was heated at 37° C. for 30 min to facilitate bacterial growth, before being subject to the hybridization assay. Each set of droplets were then detected with the LIF detector, and the resulting data was subject to histogram analysis, where a threshold (“Thresh”, FIG. 13Ai), 5 standard deviations above the mean intensity of empty droplets, was calculated for distinguishing positive droplets. In these experiments, the culture-free (FIG. 13Ai) and cultured E. coli cells (FIG. 13Aii) yield frequencies of positive droplets (7.02% and 7.70%, respectively) which correspond well with the expected 8% based on the input concentration of E. coli (4×10⁷ CFU/mL). Importantly, the mean intensity of the cultured E. coli-containing droplets is much higher than that of droplets that forewent culture, indicative of the increased production of 16S rRNA from these growing E. coli cells.

Next, we co-encapsulated the EC probe, E. coli-spiked MH broth, and gentamicin (high bactericidal concentration of 128 μg/mL) into picoliter droplets and performed 30 min culture/antibiotic exposure followed by the hybridization assay and LIF detection. When compared to the cultured no-antibiotic control, the gentamicin-dosed E. coli droplet population yields a relatively reduced frequency of positive droplets (3.12%, FIG. 13Aiii). The reduction in positive droplet frequency can be attributed to the relatively lower average fluorescence signal from gentamicin-dosed positive droplets. This decrease in fluorescence aligns well with our previous observations from bulk measurements (FIG. 10), indicating that E. coli cells in droplets produce lower quantities of 16S rRNA in the presence of the antibiotic (FIG. 13Aiii). Indeed, the normalized positive droplet population, defined as the frequency of positive droplets with antibiotics divided by the frequency of positive droplets without antibiotics, decreases as we increase the concentration of antibiotics, indicative of an antimicrobial dose-dependent response of bacterial 16S rRNA production (FIG. 14).

We next sought to minimize the culture/antibiotic exposure time for assessing antimicrobial susceptibility in urine. We separately digitized reference and multi-drug resistant E. coli-spiked urine samples, each along with 2×MH broth and EC PNA probes in droplets without and with gentamicin (bactericidal concentration of 8 μg/mL). Each gentamicin-free and gentamicin-dosed droplet group pair was then incubated at 37° C. for 30, 15, 10, or 5 min, followed by lysis, hybridization, and LIF detection (FIG. 13B). Longer bacterial culture result in higher production of 16S rRNA in gentamicin-free bacterial cells and therefore increased positive droplet frequencies. At the same time, gentamicin restricts the production of 16S rRNA in gentamicin-dosed reference E. coli cells, reflected by the decreasing normalized positive droplet population over increasing culture times (FIG. 13B, green). In contrast, gentamicin has a significantly lesser effect on 16S rRNA production for the resistant E. coli cells, reflected by the comparatively higher and relatively unchanging normalized positive droplet population (FIG. 13B, red). The effect of gentamicin on the two strains can be differentiated after as short as 10 min culture/antibiotic exposure, the minimum time at which there is no overlap between the error envelopes around the normalized positive droplet populations. This result highlights the benefit of single-cell measurements of 16S rRNA via droplet microfluidics, where even with drastically shortened culture times, by processing a large number of droplets at a high throughput (˜kHz), we sample sufficient single cells that are representative of the entire population to allow us to differentiate susceptible and resistant bacteria. As a result, we achieve assessment of antimicrobial susceptibility faster than the average replication time of an E. coli cell, a feat that ultimately paves the way for integrating molecular ID and AST to well under 30 min.

Finally, we investigated the utility of our single-cell 16S rRNA-based phenomolecular AST approach against 3 clinically important antibiotics. We tested the reference and resistant strains of E. coli against 3 antibiotics from distinct classes with distinct mechanisms of action at relevant concentrations guided by the Clinical Laboratory Standards Institute (CLSI) (63): 8 μg/mL gentamicin (an aminoglycoside that inhibits protein synthesis), 0.5 μg/mL ciprofloxacin (a fluoroquinolone that inhibits DNA gyrase activity), and 16 μg/mL ampicillin (a beta-lactam that inhibits cell wall synthesis). For both gentamicin and ciprofloxacin, our platform can differentiate antibiotic-susceptible from antibiotic-resistant E. coli after only 10 min of culture/antibiotic exposure. For ampicillin, 60 min of culture/antibiotic exposure is required to effectively differentiate the two E. coli strains (FIG. 13C). These results show the utility of our method for achieving reliable assessment of antimicrobial susceptibility for 3 different antibiotics with distinct mechanisms of action, though the total assay time may be dependent on the particular antibiotic chosen. For UTIs, our results are especially promising because gentamicin and ciprofloxacin are commonly administered oral and parenteral antibiotics, indicating the potential of our approach as a rapid screening tool for safeguarding the use these antibiotics.

### Clinical Comparison Study of DropDx for Rapid Bacterial ID Classification and Antimicrobial Susceptibility Assessment From 50 Clinical Urine Specimens

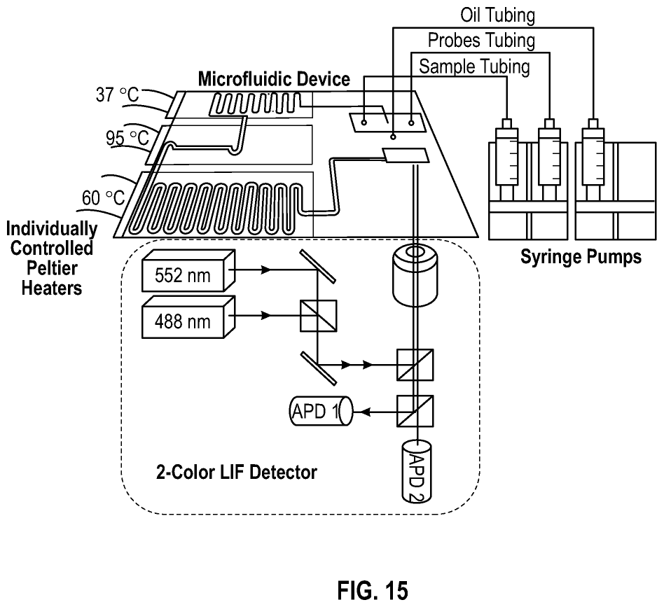

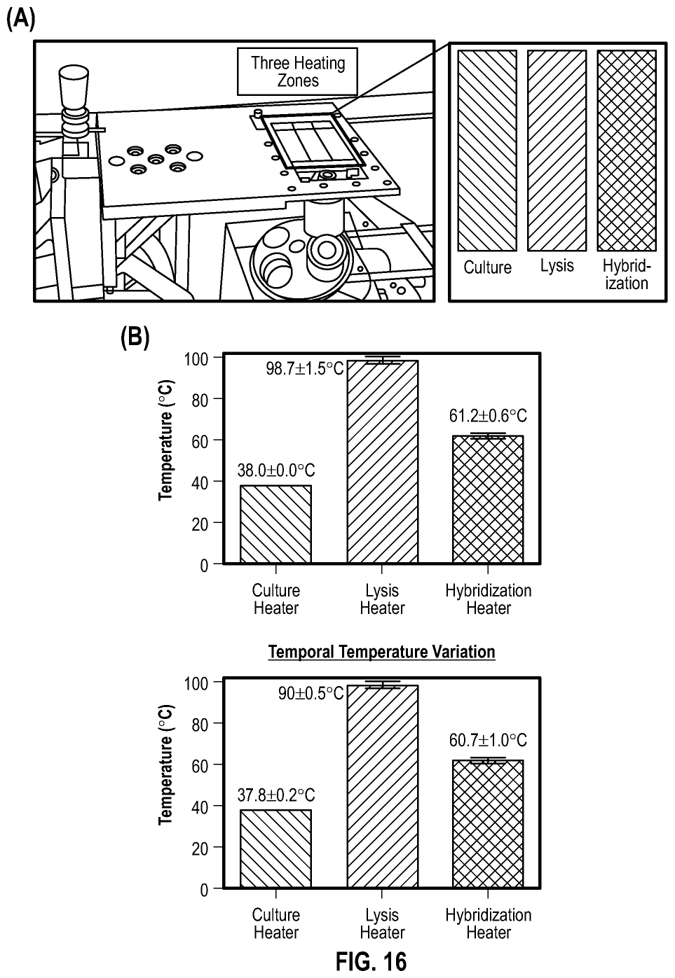

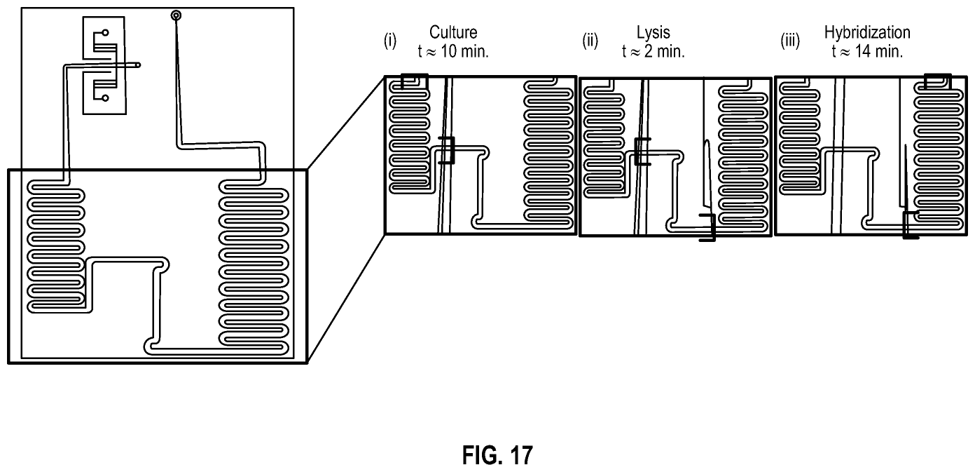

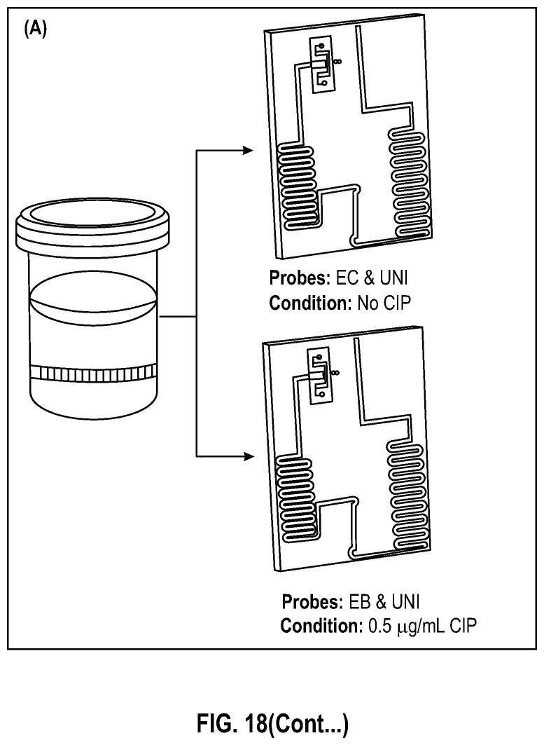

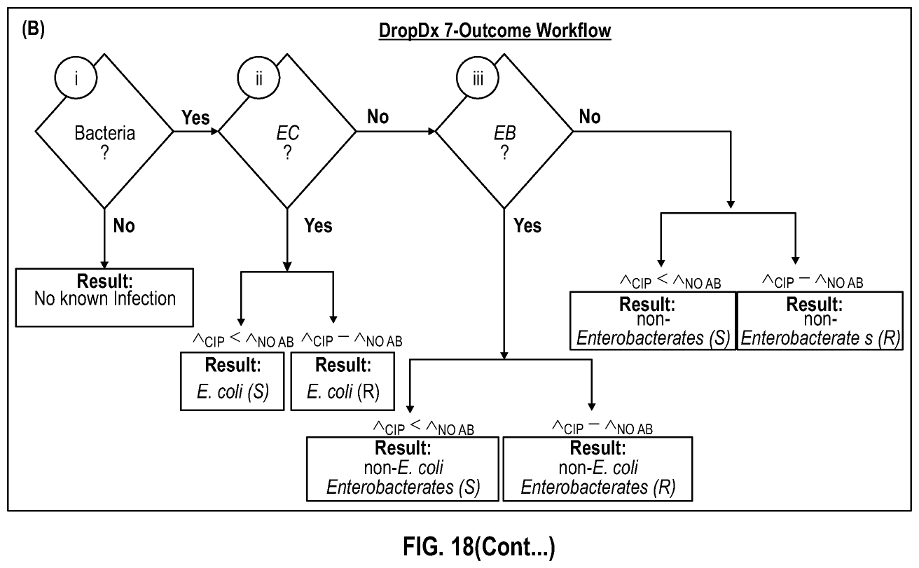

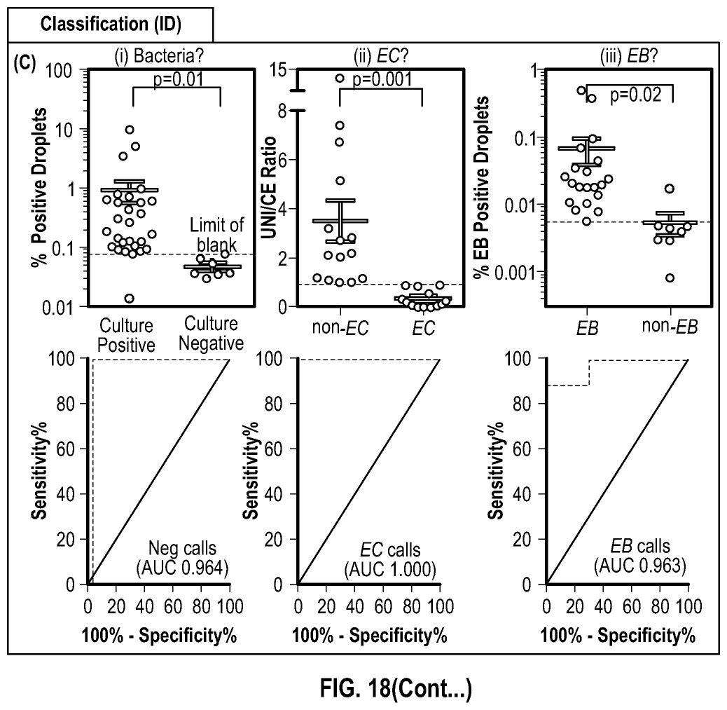

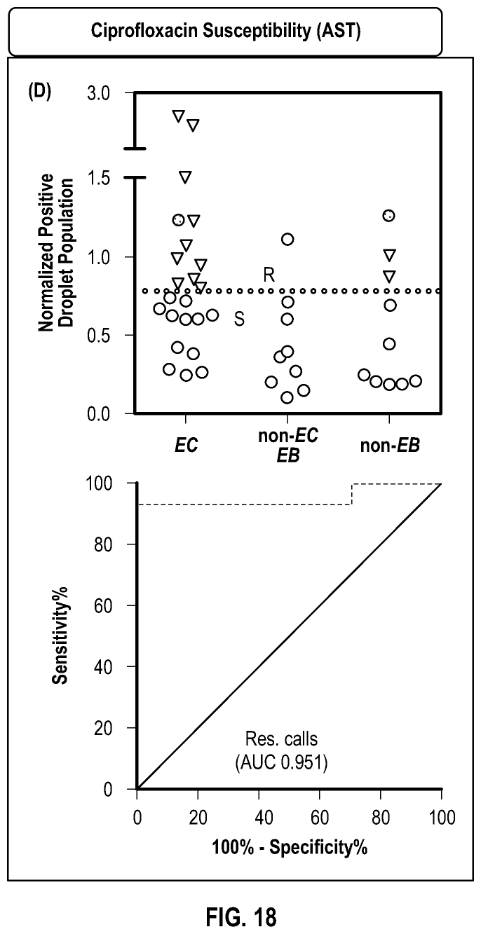

Prior to clinical validation with patient urine specimens, we first constructed the fully integrated platform, DropDx, for automated and hands-free operation from sample input to bacterial diagnosis. We then devised a simple yet useful workflow to classify 7 unique UTI diagnostic outcomes that cover ˜90% UTI cases and a front-line antibiotic ciprofloxacin using DropDx. To do so, we designed and fabricated a monolithic DropDx device, positioned its incubation channel above 3 individually controlled Peltier heaters kept at either 37° C. for culture/antibiotic exposure, 95° C. for lysis, or 60° C. for hybridization (FIG. 15), and finally aligned its detection channel to the 2-color LIF detector for fluorescence measurements. We experimentally determined that the droplets generated in the DropDx device flowed continuously through the 37° C. segment in ˜10 min, the 95° C. segment in ˜2 min, and the 60° C. segment in ˜14 min (FIG. 16 and FIG. 17), resulting in an assay time of ˜26 min. We then designed our DropDx workflow using 2 DropDx devices that can be used in parallel to achieve uropathogen ID/classification and AST against ciprofloxacin. This was accomplished by analyzing each urine specimen with FAM-labeled EC probe, Alexa546-labeled UNI probe, and no ciprofloxacin in the first device, and analyzing the same specimen with FAM-labeled EB probe, Alexa546-labeled UNI probe, and 0.5 μg/mL ciprofloxacin in the second device (FIG. 18A). Here, we chose to work with ciprofloxacin because this commonly administered broad-spectrum antibiotic for UTIs is becoming increasingly ineffective due to emerging resistance and therefore can be safeguarded with a diagnostic tool like DropDx. A ciprofloxacin dosage of 0.5 μg/mL was chosen based on the minimum inhibitory concentration interpretive breakpoint range per the CLSI guideline (63). Using our DropDx workflow, we can determine 1 of 7 diagnostic outcomes for each urine specimen (namely, no known infection, E. coli (S), E. coli (R), non-E. coli Enterobacterales (S), non-E. coli Enterobacterales (R), other (S), and other (R))—within as little as 30 min (FIG. 18B).

Using our DropDx platform and workflow, we conducted a clinical comparison study of 50 deidentified patient urine specimens obtained from the Johns Hopkins Hospital Clinical Microbiology Laboratory. Each urine specimen was subject to the clinical standard diagnostic workflow that included clinical isolation via plating, ID via matrix-assisted laser desorption/ionization-time of flight (MALDI-TOF) mass spectrometry, and AST via BD Phoenix enabled broth microdilution testing. Clinical ID/AST reports for each specimen required at least 48 h from patient collection. DropDx was used to test refrigerated urine specimens that had freshly entered the clinical workflow, in order to obtain comparable results within a small fraction of the time.

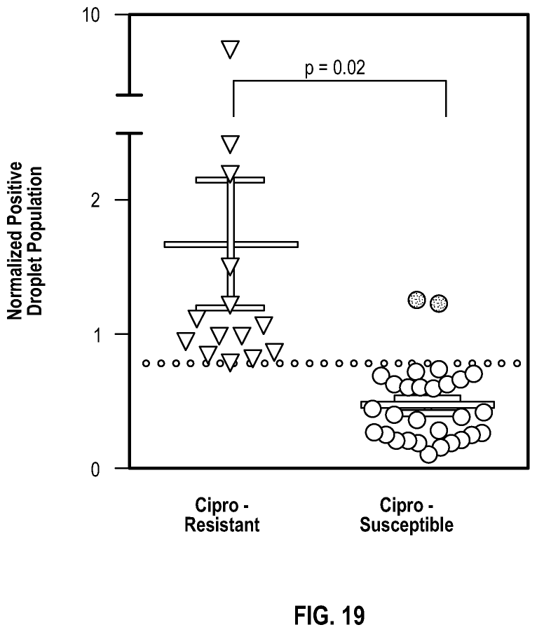

For all 50 specimens tested, DropDx delivers accurate and robust performance as a diagnostic platform for both pathogen identification and antimicrobial susceptibility assessment. A summary of all tested specimens, along with a comparison of DropDx readouts and clinical readouts, is provided in Table 3. Threshold values used to differentiate each category of calls were selected by receiver-operating curve (ROC) analysis (see Materials and Methods). DropDx can successfully differentiate culture-negative from culture-positive specimens for all but 1 of the tested urine specimens. The single false-negative call is likely due to low input concentration of bacteria or high optical background from urine (FIG. 18Ci). DropDx achieves accurate differentiation of all E. coli from non-E. coli culture-positive specimens (FIG. 185Cii), owing to the high specificity and sensitivity of the designed PNA probes. Finally, DropDx successfully differentiates all but 1 of the non-E. coli Enterobacterales from Enterobacterales specimens (FIG. 18Ciii). As a measure of diagnostic utility, ROC curves are plotted for all 3 metrics of classification, resulting in high areas-under-curve (AUC for Negative/Positive=0.964, AUC for EC/non-EC=1.000, and AUC for EB/non-EC EB=0.963. All culture-positive specimens were screened for resistance to first-line oral antibiotic ciprofloxacin. From a total of 43 culture-positive specimens screened, we observe only 2 false resistance calls (FIG. 18D). In comparing with the clinical standard AST, DropDx achieves a categorical agreement of 95.3% with no major errors, and an AUC of 0.951—a highly favorable outcome for a novel diagnostic test (64) (FIG. 19).

TABLE 3
Summary of urine samples tested and final results in clinical comparison study. For each sample tested,
a summary of the ID and AST readouts from clinical standard workflows (MALDI/TOF and BD Phoenix) is
listed against the DropDx classification and resistance call. Disagreements in data are bolded.

| | | Clinical Readout | | DropDx Readout | | |
|---|---|---|---|---|---|---|
| Internal | Sample | | Ciprofloxacin | | Ciprofloxacin | |
| Designation | Number | Pathogen ID | Resistance | Pathogen ID | Resistance | Notes |
| BA2-10182018 | 1 | E. coli | S | NA | S | AST Pilot |
| BA5-10232018 | 2 | K. pneumoniae | S | NA | S | AST Pilot |
| BA6-10232018 | 3 | E. coli | S | NA | S | AST Pilot |
| BA7-10232018 | 4 | E. coli | S | NA | R | AST Pilot |
| BA8-10232018 | 5 | P. vulgaris | S | NA | S | AST Pilot |
| BA9-10302018 | 6 | E. coli | S | NA | S | AST Pilot |
| BA14-11012018 | 7 | E. coli | S | NA | S | AST Pilot |
| BA15-11012018 | 8 | E. coli | S | NA | S | AST Pilot |
| BA16-11012018 | 9 | E. coli | R | NA | R | AST Pilot |
| BA17-11012018 | 10 | E. coli | R | NA | R | AST Pilot |
| BA18-11072018 | 11 | E. coli | S | NA | S | AST Pilot |
| BA19-11072018 | 12 | E. coli | R | NA | R | AST Pilot |
| BA21-11072018 | 13 | P. aeruginosa | S | NA | S | AST Pilot |
| BA22-11272018 | 14 | P. aeruginosa | R | NA | R | AST Pilot |
| BA23-11272018 | 15 | E. coli | S | NA | S | AST Pilot |
| BA26-12042018 | 16 | K. pneumoniae | S | Non-EC | S | ID Pilot/AST |
| | | | | EB | | Validation |
| BA29-12042018 | 17 | E. coli | S | EC | S | ID Pilot/AST |
| | | | | | | Validation |
| CC30-12122018 | 18 | S. marsescens | S | Non-EC | S | ID Pilot/AST |
| | | | | EB | | Validation |
| BA31-12112018 | 19 | E. coli | S | EC | S | ID Pilot/AST |
| | | | | | | Validation |
| BA34-12112018 | 20 | Negative | NA | Negative | NA | ID Pilot/AST |
| | | | | | | Validation |
| BA38-12132018 | 21 | E. cloacae | S | Non-EC | S | ID Pilot/AST |
| | | | | EB | | Validation |
| BA41-12132018 | 22 | Negative | NA | Negative | NA | ID Pilot/AST |
| | | | | | | Validation |
| BA42-12182018 | 23 | E. coli | R | EC | R | ID Pilot/AST |
| | | | | | | Validation |
| BA48-12202018 | 24 | K. oxytoca/R. | S | Non-EC | S | ID Pilot/AST |
| | | orinthinolytica | | EB | | Validation |
| BA49-12202018 | 25 | E. coli | S | EC | S | ID Pilot/AST |
| | | | | | | Validation |
| BA58-01032019 | 26 | Negative | NA | Negative | NA | ID Pilot/AST |
| | | | | | | Validation |
| BA63-01102019 | 27 | Negative | NA | Negative | NA | ID Pilot/AST |
| | | | | | | Validation |
| BA85-02192019 | 28 | P. aeruginosa | S | Non-EB | S | ID Pilot/AST |
| | | | | | | Validation |
| BA86-02192019 | 29 | P. aeruginosa | S | Negative | R | ID Pilot/AST |
| | | | | | | Validation |
| BA90-02142019 | 30 | P. aeruginosa | S | Non-EB | S | ID Pilot/AST |
| | | | | | | Validation |
| BA54-01032019 | 31 | P. aeruginosa | S | Non-EB | S | ID Pilot/AST |
| | | | | | | Validation |
| BA57-01032019 | 32 | K. pneumoniae | S | Non-EC | S | Validation |
| | | | | EB | | |
| BA60-01102019 | 33 | E. coli | S | EC | S | Validation |
| BA61-01102019 | 34 | E. coli | S | EC | S | Validation |
| BA64-01102019 | 35 | Negative | NA | Negative | NA | Validation |
| BA68-01172019 | 36 | Negative | NA | Negative | NA | Validation |
| BA76-01242019 | 37 | Negative | NA | Negative | NA | Validation |
| BA91-02142019 | 38 | P. aeruginosa | S | Non-EB | S | Validation |
| BA92-02212019 | 39 | P. aeruginosa | S | Non-EB | S | Validation |
| BA93-02212019 | 40 | P. aeruginosa | S | Non-EB | S | Validation |
| BA97-03142019 | 41 | P. mirabilis | R | Non-EC | R | Validation |
| | | | | EB | | |
| BA98-03282019 | 42 | P. aeruginosa | R | Non-EC | R | Validation |
| | | | | EB | | |
| BA100-03282019 | 43 | E. coli | R | EC | R | Validation |
| BA101-04042019 | 44 | E. coli | R | EC | R | Validation |
| BA104-04112019 | 45 | E. coli | R | EC | R | Validation |
| BA107-05072019 | 46 | E. coli | R | EC | R | Validation |
| BA108-05072019 | 47 | E. coli | R | EC | R | Validation |
| BA111-05282019 | 48 | E. coli | R | EC | R | Validation |
| BA112-05312019 | 49 | C. freundii | S | Non-EC | S | Validation |
| | | | | EB | | |
| BA113-05312019 | 50 | E. coli | R | EC | R | Validation |

### Discussion

We introduce DropDx, the first UTI diagnostic platform that leverages droplet microfluidics for single-cell measurements of bacterial 16S rRNA to enable unprecedented pathogen ID/classification and AST from urine in as little as 30 min. The development of DropDx was achieved by implementing 3 enabling concepts synergistically within an integrated platform. First, employment of 16S rRNA as a novel molecular surrogate for phenotypic bacterial response to antibiotics in the emerging approach of pheno-molecular AST allows us to achieve both pathogen ID/classification and AST in a one-step assay. Second, confinement of highly abundant 16S rRNA from single bacterial cells within picoliter droplets makes their detection via hybridization-based probes possible, thus enabling rapid and simple detection of bacteria without nucleic acid amplification and minimal sample preparation. Finally, high-throughput and quantitative measurements of 16S rRNA from hundreds to thousands of single bacterial cells via droplets offer a new and powerful approach for shortening culture/antibiotic exposure and accelerating pheno-molecular AST.

In this example, we tailor DropDx to facilitate a useful yet practical uropathogen ID/classification scheme that can improve treatments for UTI. For pathogen ID in UTIs, rapid binary detection of a single predominant bacterial species such as E. coli is useful but nevertheless limited. On the other hand, broad-based species-level ID would undoubtedly be ideal but would require the use of additional devices or additional assay steps, both of which can increase operational complexity and turnaround time. We instead focus on 4 ID/classification categories that account for ˜90% of UTI cases: whether or not a Gram-negative bacterial infection is present and whether it is E. coli, in the Enterobacterales order, or a different Gram-negative bacteria. Because 16S rRNA is well-characterized for determining bacterial phylogeny for species-level, order-level, and kingdom-level bacterial ID, we only need 3 detection probes to cover the 4 ID/classification categories. Moreover, 2-color detection in DropDx allows us to achieve the necessary multiplexability to cover ˜90% of UTIs, using only 2 devices. An example case of the usefulness of our ID/classification scheme is when DropDx identifies a non-Enterobacterales that is ciprofloxacin-resistant (as with specimen #42), a physician could immediately prescribe the most appropriate alternative antibiotic based on the antibiotic resistance rates for non-Enterobacterales in the community.

Our results show that, by using only the pan-bacteria UNI probe to quantitatively measure 16S rRNA from single bacterial cells in picoliter droplets, reliable AST was achieved for 9 bacterial species against ciprofloxacin. Such single-cell measurements of 16S rRNA also achieve reliable AST for E. coli against gentamicin, ciprofloxacin, and ampicillin. These results suggest that, whereas multiple species- and/or antibiotic-dependent mRNA sequences have been required to achieve reliable AST in other approaches (65), 16S rRNA can potentially serve as a broad surrogate for pheno-molecular AST with less dependence on bacterial species and antibiotics. This is perhaps because 16S rRNA is an integral structural building block of ribosomes that is replicated during growth and doubling of bacterial cells, which is independent from the specific inhibitory mechanisms of the tested antibiotics. While the demonstrated combinations of bacteria and antibiotics in DropDx show great promises, further validation of 16S rRNA in pheno-molecular AST with more bacterial species against additional classes of antibiotics (such as sulfonamides and polymyxins) is needed.

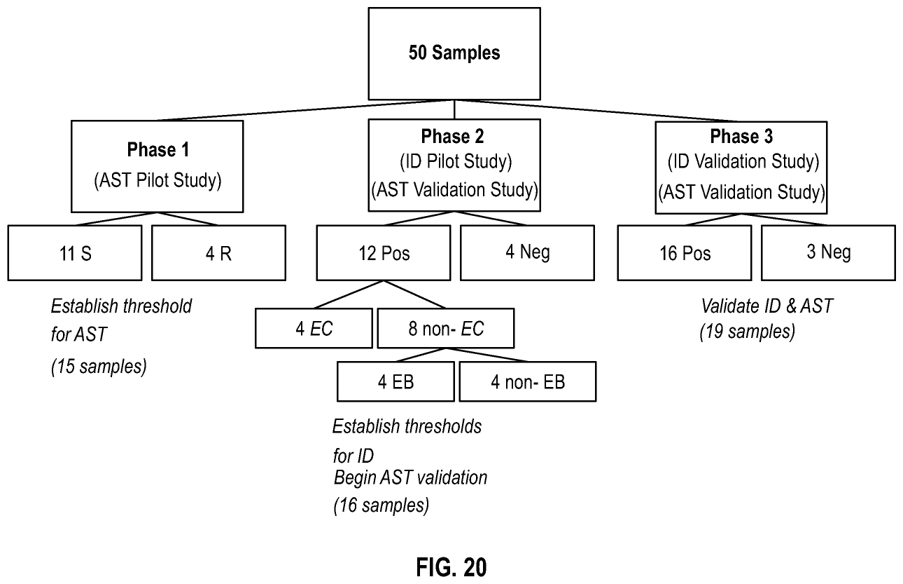

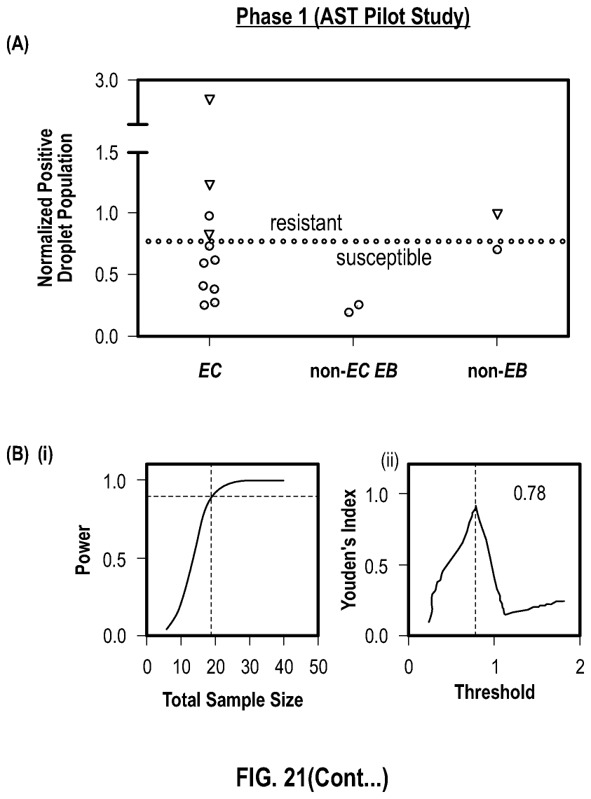

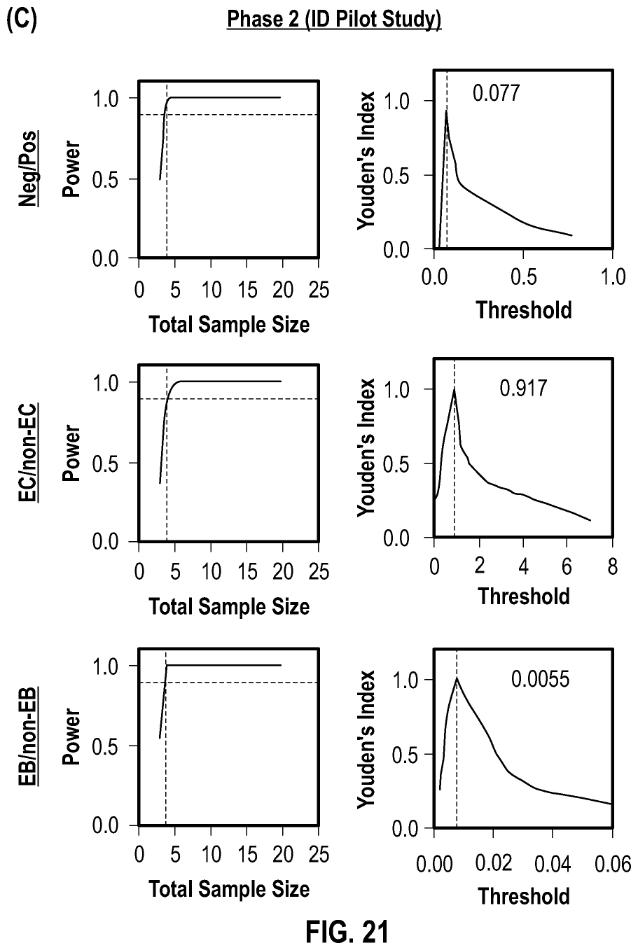

Clinically oriented evaluation of new diagnostic technologies should ideally include both a pilot phase and a validation phase that is unbiased, akin to clinical trials (66). However, such a two-phased study design has not been a common practice for developing microfluidic-based diagnostic technologies. We in fact have followed such a study design as we evaluated ID and AST performances of DropDx (FIG. 20). Among the 50 patient urine specimens tested in this work, we used the initial 15 for AST pilot study (FIG. 17, Phase 1), the next 16 concurrently for ID pilot study and AST validation study (FIG. 17, Phase 2), and the final 19 for both ID validation study and AST validation study (FIG. 17, Phase 3). Of the 35 specimens in Phases 2 and 3, 28 were positive for bacterial infections and eligible for AST validation. We used the specimens in the pilot phases to (1) perform ROC analysis and find the maximum Youden's Index (67-69) for determining the optimal ID and AST thresholds that would be applied, without bias, to all subsequent specimens in the validation phases, and to (2) conduct power analysis to ensure that sufficient specimens are enrolled for validation. The threshold established from the 15 specimens for calling the susceptibility/resistance to ciprofloxacin in Phase 1 was validated as it unbiasedly called the susceptibility/resistance to ciprofloxacin for 27 of the 28 eligible specimens in Phases 2 and 3. Power analysis in Phase 1 indicates that the 28 specimens tested in in Phases 2 and 3 was sufficient, as only 20 samples were required to obtain a statistical power of 90% and confidence of 95% (FIG. 21). Similarly, the 3 thresholds for calling bacterial infection, E. coli, and Enterobacterales that were established from the 16 samples in Phase 2 were validated as they successfully classified 18 out of 19 samples in Phase 3. Power analyses in Phase 2 show that statistical power of 90% and confidence of 95% for these 3 cases could be achieved by testing 4 samples in Phase 3 (FIG. 21), which we again exceeded. These results show that DropDx is robust enough to withstand rigorous evaluation that includes a pilot phase and an unbiased validation phase.

In some embodiments, more antibiotics are incorporated into DropDx to generate a more complete antibiogram, which is useful for both diagnostics and surveillance of antimicrobial resistance. Similarly, an enlarged panel of fluorogenic PNA probes is utilized in certain embodiments to broaden the pathogen ID/classification capacity. This can be accomplished, for example, by implementing LIF detectors that can detect 3 or 4 fluorescence colors or by using a multiplexed droplet device (70) that can test multiple groups of droplets. In some embodiments, DropDx is expanded to detect Gram-positive uropathogens. In certain of these embodiments, the assays are modified with the addition of chemical lysing agents and/or cyclic thermal shocking to access their 16S rRNA or the addition of viability dyes (e.g., resazurin) (59, 71) that non-specifically detect any microorganism. DropDx has shown great potential for clinical utility, underscored by its amplification-free approach and urine sample-to-answer workflow.

While the foregoing disclosure has been described in some detail by way of illustration and example for purposes of clarity and understanding, it will be clear to one of ordinary skill in the art from a reading of this disclosure that various changes in form and detail can be made without departing from the true scope of the disclosure and may be practiced within the scope of the appended claims. For example, all the methods, devices, systems, computer readable media, and/or component parts or other aspects thereof can be used in various combinations. All patents, patent applications, websites, other publications or documents, and the like cited herein are incorporated by reference in their entirety for all purposes to the same extent as if each individual item were specifically and individually indicated to be so incorporated by reference.

1. A method of identifying and determining an antimicrobial susceptibility of bacteria in a sample, the method comprising:
forming at least one set of droplets that each comprise at least one bacterial cell from the sample, at least one antimicrobial agent, and at least one set of probes, wherein the set of probes comprises a plurality of probes that each comprise at least one nucleobase sequence that is at least partially complementary to at least one region of at least one 16S ribosomal RNA (rRNA) sequence of 16S rRNA from the bacterial cell and wherein the plurality of probes each comprise at least one reporter moiety and at least one quencher moiety that substantially quenches the reporter moiety at least when the plurality of probes are not hybridized to the 16S rRNA;
incubating the set of droplets for a first time period under conditions sufficient for the bacterial cells in the droplets to multiply and/or to at least produce additional 16S rRNA if the bacterial cells are not susceptible to the antimicrobial agent, or to not substantially multiply and/or to not substantially produce additional 16S rRNA if the bacterial cells are susceptible to the antimicrobial agent;
incubating the set of droplets for a second time period under conditions sufficient for one or more of the plurality of probes to hybridize with the 16S rRNA from the bacterial cells, and optionally, to artificially amplify the 16S rRNA from the bacterial cells;
detecting a detectable signal from the reporter moiety of one or more of the probes in one or more of the droplets, thereby identifying the bacteria; and,
comparing a strength of the detectable signal from the reporter moiety with a strength of a reference detectable signal, wherein when the strength of the detectable signal from the reporter moiety is greater than the strength of the reference detectable signal, the bacterial cell is indicated not to be susceptible to the antimicrobial agent, and wherein when the strength of the detectable signal from the reporter moiety is less than the strength of the reference detectable signal, the bacterial cell is indicated to be susceptible to the antimicrobial agent, thereby determining the antimicrobial susceptibility of bacteria in the sample.

2.-6. (canceled)

7. The method of claim 1, wherein the sample comprises a urine sample.

8. (canceled)

9. The method of claim 1, comprising obtaining the sample from at least one subject.

10. (canceled)

11. The method of claim 1, comprising administering one or more antimicrobial therapeutic agents to the subject upon identifying and determining the antimicrobial susceptibility of the bacteria in the sample.

12. The method of claim 1, wherein the plurality of droplets each comprise a single bacterial cell from the sample.

13. The method of claim 1, wherein the antimicrobial agent comprises an antimicrobial therapeutic agent.

14.-21. (canceled)

22. The method of claim 1, wherein the set of probes is selected from Table 1.

23.-25. (canceled)

26. The method of claim 1, wherein the reference detectable signal is obtained from droplets that comprise the bacterial cell and the set of probes and lack the antimicrobial agent.

27. The method of claim 1, wherein a given droplet comprises a volume of about 10 picoliters (pL) or less.

28. The method of claim 1, wherein a given droplet comprises between about 10³ and about 10⁵ copies of the rRNA.

29. The method of claim 1, wherein the first time period comprises about 15 minutes or less.

30. The method of claim 1, comprising incubating the set of droplets for the first time period at a temperature of about 37° C.

31. The method of claim 1, wherein the second time period comprises about 20 minutes or less.

32. The method of claim 1, comprising incubating the set of droplets for the second time period at a temperature of about 60° C.

33. The method of claim 1, comprising lysing the bacterial cells in the droplets.

34. The method of claim 33, comprising incubating the set of droplets for about two minutes at a temperature of about 95° C. to lyse the bacterial cells in the droplets.

35. The method of claim 1, wherein the bacterial cell comprises a Gram-negative bacterial cell.

36.-38. (canceled)

39. The method of claim 1, comprising forming the droplets in a microfluidic device.

40.-44. (canceled)

45. A device, comprising a body structure that defines at least one droplet formation cavity, at least one fluidic channel, and at least one detectable signal detection cavity,
wherein the droplet formation cavity and the detectable signal detection cavity fluidly communicate with one another via the fluidic channel,
wherein the droplet formation cavity fluidly communicates, or is capable of fluidly communicating, with at least one sample source, at least one reagent source, and at least one immiscible fluid source, which reagent source comprises at least one set of probes, wherein the set of probes comprises a plurality of probes that each comprise at least one nucleobase sequence that is at least partially complementary to at least one region of at least one ribosomal RNA (rRNA) sequence of rRNA from at least one bacterial cell and wherein the plurality of probes each comprise at least one reporter moiety and at least one quencher moiety that substantially quenches the reporter moiety at least when the plurality of probes are not hybridized to the rRNA, which droplet formation cavity is configured to form droplets that comprise at least some of the set of probes from the reagent source and at least an aliquot of a sample from the sample source,
wherein the fluidic channel comprises at least one droplet culture region, at least one bacterial cell lysis region, and at least one hybridization region, wherein the droplet culture region is configured to thermally communicate with a droplet culture thermal modulator, wherein the bacterial cell lysis region is configured to thermally communicate with a lysis thermal modulator, and wherein the hybridization region is configured to thermally communicate with a hybridization thermal modulator,
wherein the detectable signal detection cavity is configured to detectably communicate with one or more detectors that are capable of detecting detectable signals from reporter moieties of the probes when the probes hybridize with the rRNA from the at least one bacterial cell, and
wherein the device is not configured to artificially amplify nucleic acids, if present, in or from samples or the aliquot of the sample from the sample source.

46.-48. (canceled)

49. A system, comprising:
a device, comprising a body structure that defines at least one droplet formation cavity, at least one fluidic channel, and at least one detectable signal detection cavity,
wherein the droplet formation cavity and the detectable signal detection cavity fluidly communicate with one another via the fluidic channel,
wherein the droplet formation cavity fluidly communicates, or is capable of fluidly communicating, with at least one sample source, at least one reagent source, and at least one immiscible fluid source, which reagent source comprises at least one set of probes, wherein the set of probes comprises a plurality of probes that each comprise at least one nucleobase sequence that is at least partially complementary to at least one region of at least one ribosomal RNA (rRNA) sequence of rRNA from at least one bacterial cell and wherein the plurality of probes each comprise at least one reporter moiety and at least one quencher moiety that substantially quenches the reporter moiety at least when the plurality of probes are not hybridized to the rRNA, which droplet formation cavity is configured to form droplets that comprise at least some of the set of probes from the reagent source and at least an aliquot of a sample from the sample source,
wherein the fluidic channel comprises at least one droplet culture region, at least one bacterial cell lysis region, and at least one hybridization region, wherein the droplet culture region is configured to thermally communicate with a droplet culture thermal modulator, wherein the bacterial cell lysis region is configured to thermally communicate with a lysis thermal modulator, and wherein the hybridization region is configured to thermally communicate with a hybridization thermal modulator,
wherein the detectable signal detection cavity is configured to detectably communicate with one or more detectors that are capable of detecting detectable signals from reporter moieties of the probes when the probes hybridize with the rRNA from the at least one bacterial cell, and
wherein the device is not configured to artificially amplify nucleic acids, if present, in or from samples or the aliquot of the sample from the sample source;
a droplet culture thermal modulator;
a lysis thermal modulator;
a hybridization thermal modulator;
a detector that is capable of detecting detectable signals from reporter moieties of the probes when the probes hybridize with the rRNA from the at least one bacterial cell; and,
a controller operably connected to the droplet culture thermal modulator, the lysis thermal modulator, the hybridization thermal modulator, and the detector, which controller is configured to modulate temperatures of the droplet culture thermal modulator, the lysis thermal modulator, and the hybridization thermal modulator, and to effect detection of the detectable signals from the reporter moieties via the detector.

50. (canceled)
